(19) United States
(12) Patent Application Publication
(10) Pub. No.: US 2022/0257759 A1
(43) Pub. Date: Aug. 18, 2022

(54) TREATMENT OF SARCOMA USING IMMUNOMODULATION

(71) Applicant: BioXcel Therapeutics, Inc., New Haven, CT (US)

(72) Inventor: Vincent J. O'NEILL, New Haven, CT (US)

(21) Appl. No.: 17/674,371

(22) Filed: Feb. 17, 2022

Related U.S. Application Data
(60) Provisional application No. 63/150,493, filed on Feb. 17, 2021.

(51) Int. Cl. A61K 39/395; A61K 31/69; A61P 35/00

(52) U.S. Cl. CPC A61K 39/3955; A61K 2039/545; A61P 35/00; A61K 31/69

(57) ABSTRACT

The present disclosure provides a method for treating a subject afflicted with sarcoma selected from soft tissue sarcoma (such as liposarcoma, myxoid sarcoma) and osteosarcoma by administering Talabostat or a pharmaceutically acceptable salt thereof and Pembrolizumab, wherein Talabostat or a pharmaceutically acceptable salt thereof is administered at a dose of about 0.3 mg twice daily on one or more days of a treatment cycle.

### CROSS-REFERENCE TO RELATED APPLICATIONS

This application claims priority to and the benefit of U.S. Provisional Application No. 63/150,493, filed Feb. 17, 2021, the contents of which are hereby incorporated herein by reference in their entirety.

### FIELD

The present disclosure relates to a method for treating one or more sarcomas including soft tissue sarcomas and osteosarcoma, comprising administering an effective amount of Talabostat or a pharmaceutically acceptable salt thereof and therapeutically effective amount of Pembrolizumab to the subject.

### BACKGROUND

Osteosarcoma (OS) is the most prevalent aggressive malignant bone cancer affecting children and young adults, with a predilection in boys and African American descent. OS arises from primitive mesenchymal bone-forming cells and has a high propensity for lung metastasis, accounting for >80% of all OS metastatic sites. Roughly 20% of the patients presents with pulmonary metastasis (pOS) initially at diagnosis, and up to 30% of patients presenting with localized disease will relapse in distant sites, including >80% metastatic relapses in the lung. Outcome for metastatic disease remains dismal (<30%) over the last 3 decades, which accounts for almost all of OS-related mortality. OS contains complex genetic alterations, making molecular targeted therapy challenging.

Soft tissue sarcomas (STS) are a heterogeneous group of rare malignancies for which there are few effective therapies in advanced disease. Doxorubicin is considered the backbone of therapy for advanced disease. The studies supporting this indication were performed in the 1970's and 1980's; the study designs were not up to current standards and the benefits of treatment were modest. Three additional drugs have been approved in the past few years; VOTRIENT® (pazopanib), a multityrosine kinase inhibitor, for treatment of STS in patients who have received prior chemotherapy; YONDELIS® (Trabectedin), an alkylating agent for treatment of patients with unresectable or metastatic liposarcoma or leiomyosarcoma and who had previously received an anthracycline-containing regimen; and HALAVEN® (erubilin), a microtubule inhibitor, for patients with liposarcoma after treatment with an anthracycline-containing regimen. These newer agents have shown to prolong progression-free survival by several months but have no significant impact on overall survival. Thus, additional active agents would be of benefit to patients with these rare sarcomas.

### SUMMARY

The present disclosure provides that a combination comprising Talabostat or a pharmaceutically acceptable salt thereof administered at a dose of about 0.3 mg twice daily on one or more days of a treatment cycle and Pembrolizumab administered on day 1 of the treatment cycle is a safe and effective therapy to treat subjects afflicted with sarcoma such as soft tissue sarcoma and osteosarcoma. In embodiments, examples of soft tissue sarcoma include liposarcoma and myxoid sarcoma. The treatment regimen is well tolerated and produces less severe adverse effects relative to a subject with same cancer that is administered 0.6 mg once daily dose of Talabostat.

In embodiments, the present disclosure provides methods for treating one or more sarcomas in a subject in need thereof, comprising administering to said subject, Talabostat or a pharmaceutically acceptable salt thereof at a dose of about 0.3 mg twice daily on one or more days of a treatment cycle and Pembrolizumab on day 1 of the treatment cycle, wherein sarcoma is selected from soft tissue sarcoma and osteosarcoma. In embodiments, the soft tissue sarcoma is selected from liposarcoma and myxoid sarcoma. In embodiments, the sarcoma is soft tissue sarcoma. In embodiments, the sarcoma is advanced soft tissue sarcoma. In embodiments, the soft tissue sarcoma is advanced myxoid sarcoma. In embodiments, the soft tissue sarcoma is advanced liposarcoma. In embodiments, the sarcoma is metastatic.

In embodiments, the present disclosure provides methods for treating or inhibiting the growth of soft tissue sarcoma in a subject in need thereof, comprising administering to said subject, Talabostat or a pharmaceutically acceptable salt thereof at a dose of about 0.3 mg twice daily on one or more days of a treatment cycle and Pembrolizumab on day 1 of the treatment cycle. In embodiments, the soft tissue sarcoma is advanced soft tissue sarcoma.

In embodiments, the present disclosure provides methods for treating or inhibiting the growth of liposarcoma in a subject in need thereof, comprising administering to said subject, Talabostat or a pharmaceutically acceptable salt thereof at a dose of about 0.3 mg twice daily on one or more days of a treatment cycle and Pembrolizumab on day 1 of the treatment cycle.

In embodiments, the present disclosure provides methods for treating or inhibiting the growth of myxoid sarcoma in a subject in need thereof, comprising administering to said subject, Talabostat or a pharmaceutically acceptable salt thereof at a dose of about 0.3 mg twice daily on one or more days of a treatment cycle and Pembrolizumab on day 1 of the treatment cycle.

In embodiments, the method comprises at least one administration cycle (e.g. 1, 2, 3, 4, 5, 6 or more cycles), and each treatment cycle is about 21 days.

In embodiments, said treatment is administered for at least 12 weeks, or at least 24 weeks. In embodiments, said treatment is administered for at least 14 weeks, or at least 16 weeks. In embodiments, the patient achieves a complete response within 2-4 weeks after treatment.

In embodiments, the separate pharmaceutical formulations of Talabostat or a pharmaceutically acceptable salt thereof and Pembrolizumab are administered to the subject at relevant times, and in suitable amounts, during one or more treatment cycles of about 21 days, to maximize their combined immunotherapeutic effect.

In embodiments, Talabostat or a pharmaceutically acceptable salt thereof is administered orally (e.g. as a tablet formulation) at a dose of 0.3 mg twice a day.

In embodiments, Pembrolizumab is administered intravenously at a total dose of about 200 mg.

In embodiments, Talabostat or a pharmaceutically acceptable salt thereof is administered orally at a dose of about 0.2 mg twice daily on day 1-7 of the first treatment cycle followed by about 0.3 mg twice daily on day 8-14 of the first treatment cycle.

In embodiments, Talabostat or a pharmaceutically acceptable salt thereof is administered orally at a dose of about 0.3 mg twice daily on day 1-3 of the first treatment cycle followed by rest period of 4 days then about 0.3 mg twice daily on day 8-11 of the first treatment cycle.

In embodiments, Talabostat or a pharmaceutically acceptable salt thereof is Talabostat mesylate.

In embodiments, the subject afflicted with one or more sarcomas (e.g. osteosarcoma and soft tissue sarcoma such as liposarcoma, myxoid sarcoma or so on) has experienced dose-limiting toxicity (DLT) with a single 0.6 mg daily dose of Talabostat.

In embodiments, the subject has been previously treated with one or more regimens of systemic therapies for sarcoma. In embodiments, the subject has not been previously treated with any systemic therapy for sarcoma. In embodiments, the subject has experienced little or no improvement from prior systemic therapy. In embodiments, the subject has progressed with systemic therapies for sarcoma. In embodiments, the subject experienced intolerable toxicity on or after systemic therapies for sarcoma. In embodiments, the subject has relapsed after previous treatment with any systemic therapy.

In embodiments, the subject is not suffering from Ewing's sarcoma. In embodiments, the subject is not suffering from gastrointestinal stromal tumor.

In embodiments, the subject was not previously treated with PD-1/PD-L1 or CTLA-4 antibodies (or treatment naïve).

In embodiments, the subject has relapsed after treatment with PD-1/PD-L1 or CTLA-4 antibodies.

In embodiments, the subject has progressed after treatment with PD-1/PD-L1 or CTLA-4 antibodies.

In embodiments, the treatment is used alone or in combination with traditional cancer treatments, such as radiotherapy, or surgery.

In embodiments, the subject administered with such a treatment regimen achieves a stable disease response or better as measured by RECIST 1.1.

In embodiments, the subject administered with such a treatment regimen achieves a partial disease response or better, as measured by RECIST 1.1.

In embodiments, the subject administered with such a treatment regimen achieves a complete disease response, as measured by RECIST 1.1.

In embodiments, the disclosure provides a method of enhancing an innate immune response in a subject afflicted with one or more sarcomas (e.g. an advanced sarcoma) selected from osteosarcoma and soft tissue sarcoma (for example, liposarcoma and myxoid sarcoma) comprising administering to said subject, Talabostat or a pharmaceutically acceptable salt thereof at a dose of about 0.3 mg twice daily on one or more days of a treatment cycle (e.g. day 1 to 14) and Pembrolizumab on day 1 of the treatment cycle, wherein the enhanced innate immune response is associated with increased tumoricidal natural killer cells and macrophages, as well as the activity of NK cells and CD8+ T cells. In embodiments, the enhanced innate immune response is associated with suppression of T-regulatory cells. In embodiments, the sarcoma is liposarcoma. In embodiments, the sarcoma is a soft tissue sarcoma. In embodiments, the sarcoma is myxoid sarcoma.

In embodiments, the sarcoma is soft tissue sarcoma. In embodiments soft tissue sarcoma is advanced soft tissue sarcoma. In embodiments, the soft tissue sarcoma is myxoid sarcoma.

In embodiments, the present disclosure provides a method of enhancing proinflammatory cytokine release in a subject afflicted with one or more sarcomas selected from osteosarcoma and soft tissue sarcoma (for example, liposarcoma and myxoid sarcoma), the method comprising administering to said subject, Talabostat or a pharmaceutically acceptable salt thereof at a dose of about 0.3 mg twice daily on one or more days of a treatment cycle and Pembrolizumab on day 1 of the treatment cycle, wherein the enhanced proinflammatory cytokine release is associated with activation of Natural Killer (NK) cells and T cells resulting in increased antitumor response.

In embodiments, the proinflammatory cytokines are one or more of IL-18, IL-1β and IFN-γ. In embodiments, the subject experiences an increase in pro-inflammatory cytokines relative to a subject that is administered a single 0.6 mg daily dose of Talabostat or a pharmaceutically acceptable salt thereof.

In embodiments, the present disclosure provides a method of reducing the treatment-related adverse events (TRAEs) in a subject afflicted with one or more sarcomas (e.g. an advanced sarcomas) selected from osteosarcoma and soft tissue sarcoma (for example, liposarcoma and myxoid sarcoma), comprising administering to said subject, Talabostat or a pharmaceutically acceptable salt thereof at a dose of about 0.3 mg twice daily on one or more days and Pembrolizumab on day 1 of a treatment cycle. In embodiments, the subject experiences less treatment-related adverse events (TRAEs) or no DLT relative to a subject with the same sarcoma that is administered a single 0.6 mg once daily dose of Talabostat.

In embodiments, the TRAEs are selected from one or more of hypotension, dizziness, headache, syncope, dyspnea, chills, pyrexia, malaise, weakness, edema/peripheral swelling, hypovolemia, hypothermia, fatigue, nausea, vomiting, diaphoresis, flushing, migraine, diarrhea, constipation, alopecia, pharyngitis, chest pain, anorexia, weight increase, weight decrease, vertigo, syncope, conjunctivitis, blurred vision, pallor, pruritus, rash, fungal vaginosis, hyperglycemia, hyperkalemia, hypokalemia, hoarseness, dyspnea, anoxia, deep venous thrombosis, upper respiratory infection, blood in stool, dizziness, rigors, sepsis, pain, hypereosinophilia, dehydration, electrolyte imbalance, arthralgia, rhabdomyolysis, myalgia, constipation, hypocalcemia, neutropenia, febrile neutropenia, anemia, leukopenia, pancytopenia, and lymphopenia, somnolence, insomnia, epistaxis, dyspepsia, dysgeusia, thrombocytopenia, cyanosis peripheral, hypovolemic shock, respiratory failure, cough, pneumonitis, cardiac tamponade, acidosis, renal failure and cardiac arrest.

In embodiments, the subject experiences no TRAEs. In embodiments, the subject experiences TRAEs that are consistent with cytokine release.

In embodiments, the sarcoma is selected from liposarcoma, soft tissue sarcoma and, myxoid sarcoma. In embodiments, the sarcoma is soft tissue sarcoma. In embodiments, the sarcoma is myxoid sarcoma. In embodiments, the sarcoma is liposarcoma.

Other features, objects, and advantages of the disclosure will be apparent from the description and drawings, and from the claims.

### BRIEF DESCRIPTION OF THE DRAWINGS

FIG. 1: shows the study scheme according to Example 1.

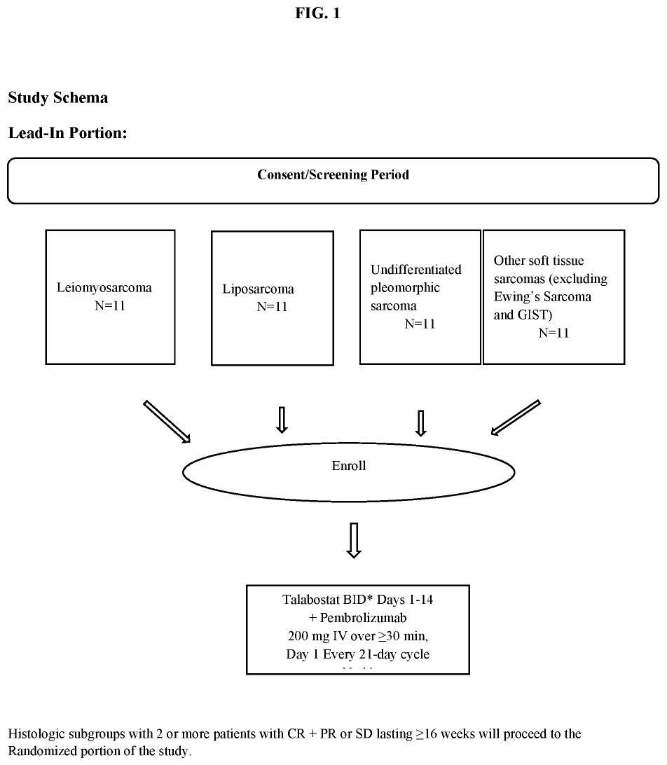

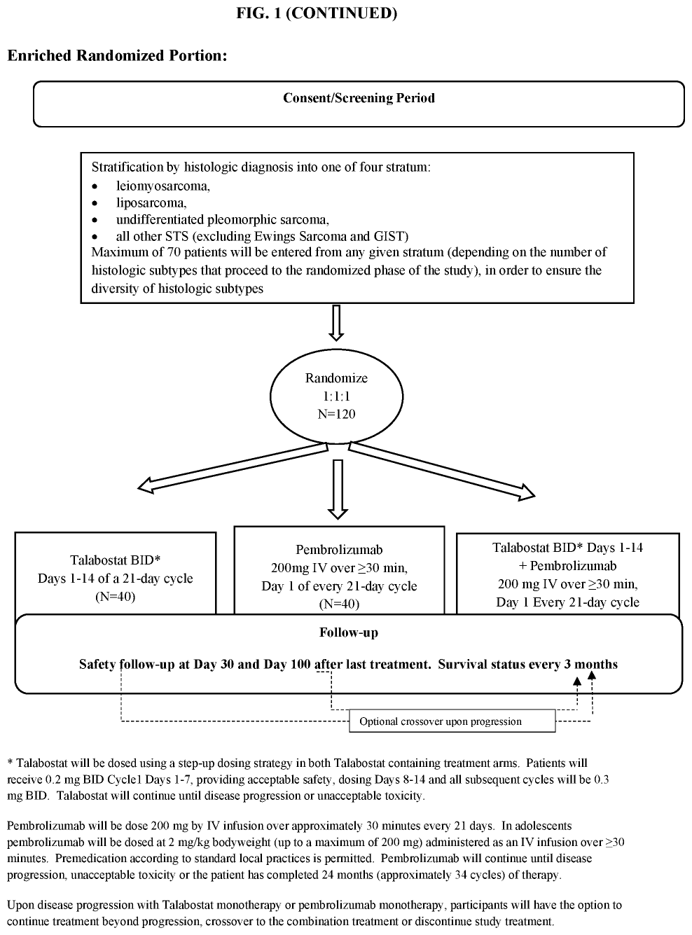

### DETAILED DESCRIPTION

In the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Abbreviations:

As used herein, the following abbreviations have the following meanings:

A2AR: A2A adenosine receptor
ALT: Alanine aminotransferase
ANC: Absolute neutrophil count
AST: Aspartate aminotransferase
AUC: Area under the plasma concentration-time curve
BS: Bone scintigraphy
BUN: Blood urea nitrogen
CAF: Cancer associated fibroblast
CR: Complete response
CT: Computed tomography
CTC: Circulating tumor cells
ctDNA: Circulating tumor DNA
CTLA4: Cytotoxic T-lymphocyte associated protein 4
DPP: Dipeptidyl peptidase
DLT: Dose limiting toxicity
DOR: Duration of response
DSRC: Data Safety Review Committee
EOT: End of Treatment
FAP: Fibroblast activation protein
GM-CSF: Granulocyte-macrophage colony-stimulating factor
G-CSF: Granulocyte-colony stimulating factor
ICI: Immune check point inhibitor
IL: Interleukin
irCR: Immune-related complete disease
irPR: Immune-related partial response
irSD: Immune-related stable disease
iRECIST: Immune Response Evaluation Criteria In Solid tumors
ITT: Intent-to-Treat
MRI: Magnetic resonance imaging
NK: Natural killer
NCI CTCAE: National Cancer Institute Common Terminology Criteria for Adverse Events
OS: Overall survival
PD: Progressive disease
PD-1: Programmed cell death 1
PD L1: Programmed death ligand 1
PD L2: Programmed death ligand 2
PFS: Progression-free survival
PR: Partial response
PD-1: Programmed Cell Death 1
Q.D: Quaque die
QTcB: QT interval corrected for heart rate using Bazett's formula
RECIST: Response Evaluation Criteria In Solid Tumors
SD: Stable disease
SAE: Serious adverse event
sHASEGP: Soluble neutral-active hyaluronidase glycoproteins
STS: Soft tissue sarcoma
Treg: Regulatory T cells or T-regulatory cells
TRAE: Treatment related adverse events
ULN: Upper limit of normal

The therapeutic agents Talabostat and Pembrolizumab as intended for use in the present disclosure are described below:

### Therapeutic Agents

Talabostat or a pharmaceutically acceptable salt thereof: Talabostat is referred to interchangeably as PT-100, Talabostat (USAN), and [(2R)-1-[(2S)-2-amino-3-methyl-1-oxobutyl]-2-pyrrolidinyl] boronic acid. Talabostat has a CAS registration number of 149682-77-9 and PubChem ID 6918572). Talabostat, also known as Val-boro-pro (L-valinyl-L-boroproline), is disclosed in PCT Appl. Publication No. 1989/003223. The IUPAC name of talabostat is [(2R)-1-[(2S)-2-amino-3-methylbutanoyl]pyrrolidin-2-yl]boronic acid. In embodiments, the Talabostat is provided as a pharmaceutically acceptable salt, such as, for example, talabostat mesylate (PubChem CID: 11522448). In embodiments, the Talabostat is the free base. In embodiments, the Talabostat or a pharmaceutically acceptable salt thereof may be a solvate. In most clinical formulations, Talabostat is provided as a salt form, e.g. Talabostat mesylate. Talabostat has two chiral centers with a R, S configuration. Talabostat or a pharmaceutically acceptable salt thereof can exist as both linear and cyclic forms (RJ Snow et al., J. Am. Chem. Soc., 1994, 116 (24), pp 10860-10869).

Talabostat or a pharmaceutically acceptable salt thereof is effective for the treatment of cancer by modulating multiple intracellular and extracellular dipeptidyl peptidases. More specifically, intracellular and extracellular dipeptidyl peptidases comprise Fibroblast Activation Protein, DPP 8/9, CD26/DPP4 and DPP2. Talabostat or a pharmaceutically acceptable salt thereof has a dual mechanism of action which includes stromal targeted activity via FAP inhibition and targeted immunostimulatory activity via DPP 8/9 inhibition. Talabostat inhibits FAP enzymatic activity thereby suppressing tumor growth. Talabostat or a pharmaceutically acceptable salt thereof also inhibits DPP8/9 thereby inducing an IL 1β response (via caspase-1) in the stroma of tumor and lymph nodes. Talabostat's dual mechanism of action introduces a novel approach to the treatment of cancer because it combines both tumor-targeted and immune-stimulatory activity in a single agent.

Pembrolizumab: Pembrolizumab (also known as MK-3475, Lambrolizumab, KEYTRUDA®, and SCH-900475) is a humanized antibody, which targets the PD-1 receptor of lymphocytes, thereby blocking PD-1 inhibitory signal transduction. Pembrolizumab may be readily procured from the marketplace.

### Methods of Use

The present disclosure relates to a method of treating one or more sarcomas (for e.g. advanced sarcomas) selected from soft tissue sarcoma and osteosarcoma. In embodiments, soft tissue sarcoma includes liposarcoma and myxoid sarcoma, comprising administering an effective amount of Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle and Pembrolizumab is administered on day 1 of the treatment cycle. In embodiments, administration of Talabostat or a pharmaceutically acceptable salt thereof and Pembrolizumab produces an overall enhanced anti-cancer effect, such as improved T-cell priming, increased T cell stimulation, increased infiltration of neutrophil and macrophages across tumor microenvironment, decreased tumor volume, increased activation of natural killer cells, enhanced activation of dendritic cells, synergistic increase in pro-inflammatory cytokine (IL1, IL2, IL18, IFN gamma, IL6, IL12p40, IL 15, IL 7, G-CSF and GM-CSF), enhanced anti-tumor memory response, reduced metastasis and reduced toxicity.

In embodiments, the present disclosure provides a method of treating a subject afflicted with one or more sarcomas (e.g. an advanced sarcomas) selected from osteosarcoma and soft tissue sarcoma comprising administering Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle and Pembrolizumab is administered on day 1 of the treatment cycle, wherein the method comprises one or more treatment cycles and each cycle of about 21 days duration. In embodiments, the soft tissue sarcoma includes liposarcoma and myxoid sarcoma.

In embodiments, administration of Talabostat or a pharmaceutically acceptable salt thereof and Pembrolizumab results in curtailment of the progression of sarcomas, reduction in tumor burden, reduced metastasis and/or inducement of tumor regression in the subject.

In embodiments, the present disclosure provides methods for treating or inhibiting the growth of soft tissue sarcoma in a subject in need thereof, comprising administering to said subject Talabostat or a pharmaceutically acceptable salt thereof at a dose of about 0.3 mg twice daily on one or more days of a treatment cycle and Pembrolizumab on day 1 of the treatment cycle.

In embodiments, the present disclosure provides methods for treating or inhibiting the growth of soft tissue sarcoma in a subject in need thereof, comprising administering to said subject Talabostat or a pharmaceutically acceptable salt thereof orally at a dose of about 0.3 mg twice daily on day 1-3 of the first treatment cycle followed by rest period of 4 days then about 0.3 mg twice daily on day 8-11 of the first treatment cycle and Pembrolizumab intravenously at a total dose of about 200 mg.

In embodiments, the present disclosure provides methods for treating or inhibiting the growth of advanced soft tissue sarcoma in a subject in need thereof, comprising administering to said subject Talabostat or a pharmaceutically acceptable salt thereof orally at a dose of about 0.2 mg twice daily on day 1-7 of the first treatment cycle followed by about 0.3 mg twice daily on day 8-14 of the first treatment cycle and Pembrolizumab intravenously on day 1 of the treatment cycle.

In embodiments, the present disclosure provides methods for treating or inhibiting the growth of advanced liposarcoma in a subject in need thereof, comprising administering to said subject Talabostat or a pharmaceutically acceptable salt thereof at a dose of about 0.3 mg twice daily on one or more days of a treatment cycle and Pembrolizumab on day 1 of the treatment cycle.

In embodiments, the present disclosure provides methods for treating or inhibiting the growth of advanced liposarcoma in a subject in need thereof, comprising administering to said subject Talabostat or a pharmaceutically acceptable salt thereof orally at a dose of about 0.3 mg twice daily on day 1-3 of the first treatment cycle followed by rest period of 4 days then about 0.3 mg twice daily on day 8-11 of the first treatment cycle and Pembrolizumab intravenously at a total dose of about 200 mg.

In embodiments, the present disclosure provides methods for treating or inhibiting the growth of advanced liposarcoma in a subject in need thereof, comprising administering to said subject Talabostat or a pharmaceutically acceptable salt thereof orally at a dose of about 0.2 mg twice daily on day 1-7 of the first treatment cycle followed by about 0.3 mg twice daily on day 8-14 of the first treatment cycle and Pembrolizumab intravenously on day 1 of the treatment cycle.

In embodiments, the present disclosure provides methods for treating or inhibiting the growth of myxoid sarcoma in a subject in need thereof, comprising administering to said subject Talabostat or a pharmaceutically acceptable salt thereof at a dose of about 0.3 mg twice daily on one or more days of a treatment cycle and Pembrolizumab on day 1 of the treatment cycle.

In embodiments, the present disclosure provides methods for treating or inhibiting the growth of myxoid sarcoma in a subject in need thereof, comprising administering to said subject Talabostat or a pharmaceutically acceptable salt thereof orally at a dose of about 0.3 mg twice daily on day 1-3 of the first treatment cycle followed by rest period of 4 days then about 0.3 mg twice daily on day 8-11 of the first treatment cycle and Pembrolizumab intravenously at a total dose of about 200 mg.

In embodiments, the present disclosure provides methods for treating or inhibiting the growth of myxoid sarcoma in a subject in need thereof, comprising administering to said subject Talabostat or a pharmaceutically acceptable salt thereof orally at a dose of about 0.2 mg twice daily on day 1-7 of the first treatment cycle followed by about 0.3 mg twice daily on day 8-14 of the first treatment cycle and Pembrolizumab intravenously on day 1 of the treatment cycle.

In embodiments, the present disclosure provides a method of delaying or preventing the progression of tumor recurrence, tumor growth or spread of a tumor in a subject afflicted with one or more sarcomas (e.g. an advanced sarcoma) selected from osteosarcoma and soft tissue sarcoma (such as liposarcoma, myxoid sarcoma etc.) comprising administering Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle and Pembrolizumab is administered on day 1 of the treatment cycle.

In embodiments, the present disclosure provides a method for initiating, sustaining or enhancing an anti-tumor immune response in a subject afflicted with one or more sarcomas (e.g. an advanced sarcomas) selected from osteosarcoma and soft tissue sarcoma. In embodiments, soft tissue sarcoma is selected from liposarcoma and myxoid sarcoma. In embodiments, the method comprises administering Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle and Pembrolizumab is administered on day 1 of the treatment cycle, wherein the subject is afflicted with soft tissue sarcoma selected from liposarcoma and myxoid sarcoma.

In embodiments, the treatment results in a sustained response in the subject after cessation of the treatment.

In embodiments, the subject is a human. In embodiments, the subject is ≥12 years old. In embodiments, the subject is less than 12 years.

In embodiments, the subject has an ECOG performance score ≤2. In embodiments, the subject has an ECOG performance score of 1. In embodiments, the subject has an ECOG performance score of 2.

In embodiments, the subject has been previously treated with one or more regimens of systemic therapies for sarcoma.

In embodiments, the subject has not been previously treated with any systemic therapy for sarcoma. In embodiments, the subject has experienced no improvement from prior systemic therapies.

In embodiments, the subject progressed from prior systemic therapies for sarcoma. In embodiments, the subject relapsed from prior systemic therapies for sarcoma. In embodiments, the subject experienced intolerable toxicity from prior systemic therapies for sarcoma.

In embodiments, the patient is pretreated with PD-1/PD-L1 or CTLA-4 antibodies. In embodiments, the patient is PD-1/PD-L1 or CTLA-4 naïve. In embodiments, the subject relapsed or progressed after prior treatment with PD-1/PD-L1 or CTLA-4 antibodies.

In embodiments, the subject is not suffering from Ewing's sarcoma. In embodiments, the subject is not suffering from gastrointestinal stromal tumor.

In embodiments, the sarcoma is selected from the group comprising of osteosarcoma, liposarcoma, soft tissue sarcoma, and myxoid sarcoma. In embodiments, the sarcoma is soft tissue sarcoma. In embodiments, the sarcoma is advanced soft tissue sarcoma. In embodiments, the soft tissue sarcoma is liposarcoma. In embodiments, the soft tissue sarcoma is liposarcoma. In embodiments, the soft tissue sarcoma is myxoid sarcoma. In embodiments, the soft tissue sarcoma is early stage. In embodiments, the soft tissue sarcoma is late stage. In embodiments, the soft tissue sarcoma is metastatic.

In embodiments, the present disclosure provides a method for reducing the treatment related adverse events (TRAEs) in a subject afflicted with soft tissue sarcoma, comprising administering Talabostat or a pharmaceutically acceptable salt thereof and Pembrolizumab, wherein Talabostat or a pharmaceutically acceptable salt thereof is administered at a dose of about 0.3 mg twice daily on one or more days of a treatment cycle and Pembrolizumab is administered on day 1 of the treatment cycle.

In embodiments, the present disclosure provides a method for reducing the treatment related adverse events (TRAEs) in a subject afflicted with liposarcoma, the method comprising administering Talabostat or a pharmaceutically acceptable salt thereof and Pembrolizumab, wherein Talabostat or a pharmaceutically acceptable salt thereof is administered at a dose of about 0.3 mg twice daily on one or more days of a treatment cycle and Pembrolizumab is administered on day 1 of the treatment cycle.

In embodiments, the present disclosure provides a method for reducing the treatment related adverse events (TRAEs) in a subject afflicted with myxoid sarcoma, comprising administering Talabostat or a pharmaceutically acceptable salt thereof and Pembrolizumab, wherein Talabostat or a pharmaceutically acceptable salt thereof is administered at a dose of about 0.3 mg twice daily on one or more days of a treatment cycle and Pembrolizumab is administered on day 1 of the treatment cycle.

TRAEs contemplated within the scope of this invention are readily apparent to a person of ordinary skill in the art, and include, but are not limited to hypotension, dizziness, headache, syncope, dyspnea, chills, pyrexia, malaise, weakness, edema/peripheral swelling, hypovolemia, hypothermia, fatigue, nausea, vomiting, diaphoresis, flushing, migraine, diarrhea, constipation, alopecia, pharyngitis, chest pain, anorexia, weight increase, weight decrease, vertigo, syncope, conjunctivitis, blurred vision, pallor, pruritus, rash, fungal vaginosis, hyperglycemia, hyperkalemia, hypokalemia, hoarseness, dyspnea, anoxia, deep venous thrombosis, upper respiratory infection, blood in stool, dizziness, rigors, sepsis, pain, hypereosinophilia, dehydration, electrolyte imbalance, arthralgia, myalgia, constipation, hypocalcemia, neutropenia, febrile neutropenia, anemia, leukopenia, pancytopenia, lymphopenia, somnolence, insomnia, epistaxis, dyspepsia, dysgeusia, thrombocytopenia, cyanosis peripheral, hypovolemic shock, respiratory failure, cough, pneumonitis, cardiac tamponade, acidosis, renal failure and cardiac arrest. In embodiments, the subject experiences no TRAEs.

In embodiments, a subject with one or more sarcomas (e.g. an advanced sarcoma) selected from osteosarcoma and soft tissue sarcoma (for example, liposarcoma and myxoid sarcoma), experiences less TRAEs relative to a subject with the same cancer administered a single 0.6 mg daily dose of Talabostat. In embodiments, the subject experiences no TRAEs.

In embodiments, the present disclosure produces an increased innate immune response as compared to the innate immune response when the subject is administered Talabostat alone. The innate immune response may be increased by infiltration of innate immune cells, in particular macrophages into the blood, and NK-cells into the tumor. In embodiments, administration of Talabostat or a pharmaceutically acceptable salt thereof and Pembrolizumab suppresses Treg function more than administering Talabostat alone.

In embodiments, administering Talabostat or a pharmaceutically acceptable salt thereof and Pembrolizumab significantly increases the tumor infiltration of immune sub-populations, such as NK-cells and macrophages, compared to monotherapies using Talabostat or a pharmaceutically acceptable salt thereof or Pembrolizumab.

### Treatment Regimens

In embodiments, a treatment cycle is about 21-days duration and Talabostat or a pharmaceutically acceptable salt thereof is administered on each of days 1 to 14 and Pembrolizumab is administered on day 1. In embodiments, the treatment is effective for treating a subject afflicted with a sarcoma (e.g. an advanced sarcoma) selected from osteosarcoma and soft tissue sarcoma. In embodiments, the soft tissue sarcoma is selected from liposarcoma and myxoid sarcoma.

In embodiments, Pembrolizumab is administered as a single dose in the regimen of this disclosure to effectively treat a subject afflicted with a soft tissue sarcoma, e.g. a soft tissue sarcoma selected from liposarcoma, soft tissue sarcoma and myxoid sarcoma.

In embodiments, Pembrolizumab may be administered at a total dose of about 1 mg/kg to about 10 mg/kg, conveniently by injection (e.g., intravenously), most preferably as continuous infusion for 30 minutes. In embodiments, Pembrolizumab may be administered at a total dose of about 2 mg/kg. In embodiments, a suitable dose of Pembrolizumab administered intravenously in the treatment regimen of the present disclosure may conveniently be from about 100 mg to about 500 mg, e.g. about 200 mg. In embodiments, Pembrolizumab is administered intravenously at a total dose of about 200 mg on day 1 of each treatment regimen.

In embodiments, Pembrolizumab (MK-3475) is administered as a liquid medicament which comprises 25 mg/ml MK-3475, 7% (w/v) sucrose, 0.02% (w/v) polysorbate 80 in 10 mM histidine buffer pH 5.5, and the selected dose of the medicament is administered by IV infusion over a time period of about 30 minutes.

In embodiments, Talabostat or a pharmaceutically acceptable salt thereof is administered twice a day. In embodiments, Talabostat or a pharmaceutically acceptable salt thereof is administered at a dose of 0.3 mg twice a day. In embodiments, Talabostat or a pharmaceutically acceptable salt thereof is administered at a dose 0.2 mg twice a day for a sufficient time (e.g. for 1 to 30 days) followed by 0.3 mg twice a day thereafter. For example, Talabostat or a pharmaceutically acceptable salt thereof is administered at a dose 0.2 mg twice a day for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, followed by 0.3 mg twice a day thereafter (i.e. for the remainder of the treatment cycle and for each treatment cycle thereafter). In embodiments, Talabostat or a pharmaceutically acceptable salt thereof is administered at a dose of about 0.2 mg twice daily on days 1-7 of the first treatment cycle followed by about 0.3 mg twice daily on days 8-14 of the first treatment cycle. In embodiments, Talabostat or a pharmaceutically acceptable salt thereof may be administered at 0.3 mg as a morning dose (e.g. about 5 am, about 6 am, about 7 am, about 8 am, about 9 am, about 10 am, or about 11 am) and 0.3 mg as an evening dose (e.g. about 5 pm, about 6 pm, about 7 pm, about 8 pm, about 9 pm, about 10 μm, or about 11 pm).

In embodiments, each treatment cycle is from 1 to 30 days in duration. For example, each treatment cycle is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In embodiments, each treatment cycle is 21 days in duration. In embodiments, the subject is administered Talabostat or a pharmaceutically acceptable salt thereof and Pembrolizumab for one or more treatment cycles. For example, in embodiments, the subject is administered Talabostat or a pharmaceutically acceptable salt thereof and Pembrolizumab for 1, 2, 3, 4, 5, 6, 7, 8, 9, 10, 11, 12, 13, 14, 15, 16, 17, 18, 19, or 20 treatment cycles. In embodiments, the subject is administered Talabostat or a pharmaceutically acceptable salt thereof and Pembrolizumab for more than one treatment cycle.

In embodiments, the Talabostat and Pembrolizumab are administered according to an intermittent dosing regimen. The term “intermittent dosing regimen” refers to repeating cycles of drug administration in which the drug is administered on one or more consecutive days (“days on”) followed by one or more consecutive days of rest on which the drug is not administered (“days off”). The cycles may be regular, in that the pattern of days on and days off is the same in each cycle, or may be irregular. In embodiments, the cycle is 21 days and Talabostat or a pharmaceutically acceptable salt thereof is administered for 1, 2, 3, 4, 5, 6, 7, 8, 9, 10, 11, 12, 13, 14, 15, 16, 17, 18, 19, or 20 consecutive days followed by 1, 2, 3, 4, 5, 6, 7, 8, 9, 10, 11, 12, 13, 14, 15, 16, 17, 18, 19, or 20 days where the patient takes no Talabostat. In embodiments, this intermittent dosing schedule occurs multiple times within the same cycle. For example, in embodiments, the cycle is 21 days and Pembrolizumab is administered intravenously on day 1 and Talabostat or a pharmaceutically acceptable salt thereof is administered for 3 consecutive days followed by 4 days where the patient takes no Talabostat, and this 7 day pattern repeats once (i.e. no Talabostat is administered on days 15-21) or twice. In embodiments, Talabostat or a pharmaceutically acceptable salt thereof is administered orally (e.g. by tablet) on each of days 1 to 14 and Pembrolizumab is administered intravenously on day 1 and no drug is administered on days 15-21.

In embodiments, dosing is stopped for 2 weeks after 2 weeks of continuous dosing. In embodiments, dosing is stopped for 1 week after 2 weeks of continuous dosing. The dosing regimen with an interval of stopping weekly dosing can be repeated multiple times. In embodiments, dosing is stopped for 2 days after 5 days of continuous dosing. In embodiments, once daily dosing is stopped for 5 days followed by 2 days followed by once daily dosing for 5 days followed by 2 days, and so on. The dosing regimen with the interval of stopping the daily dosing can be repeated multiple times.

In embodiments, the subject experiences an increase in pro-inflammatory cytokines relative to a subject that is administered a single 0.6 mg daily dose of Talabostat. In embodiments, pro-inflammatory cytokines include, but are not limited to IL-6, IL-8, IL-18, IFN-γ, and IL-1β. In embodiments, the pro-inflammatory cytokines are one or more of IL-18 and IFN-γ.

Suitable treatment regimens for treating a human patient afflicted with one or more sarcomas selected from soft tissue sarcoma and osteosarcoma include, for example, administering to the patient Talabostat or a pharmaceutically acceptable salt thereof at a dose of 0.3 mg twice daily and therapeutically effective amount of Pembrolizumab. In embodiments, the soft tissue sarcoma is liposarcoma or myxoid sarcoma. In embodiments, the regimen comprises one or more administration cycles (e.g. 1, 2, 3, 4, 5, 6 or more cycles). In embodiments, each cycle is a period of about 21 days. In embodiments, Talabostat or a pharmaceutically acceptable salt thereof is administered orally (e.g. as tablets) on each of days 1 to 14 and Pembrolizumab is administered intravenously on day 1 where no drug is administered on days 15-21.

In embodiments, during one or more treatment cycles of about 21 days, Talabostat is administered on days 1 to 14 at a total daily dose of about 0.4 mg to about 0.6 mg and Pembrolizumab is administered on day 1 at a total dose of about 100 mg to about 500 mg per day, e.g. about 200 mg per day.

In embodiments, the daily dose of Talabostat or a pharmaceutically acceptable salt thereof may be varied over time. For example, during the first cycle (including the Lead-in Stage) Talabostat or a pharmaceutically acceptable salt thereof may be administered at a lower daily dose than during subsequent cycles (e.g. Efficacy Stage). For example, Talabostat or a pharmaceutically acceptable salt thereof may conveniently be administered during the Lead-in Stage at a daily dose of about 0.4 mg, and, if there are no side effects or other criteria preventing further treatment, the subject is allowed to enter the Efficacy Stage and administered in divided doses, during Efficacy Stage, a total daily dose of about 0.6 mg. In embodiments, the Lead-in Stage and the Efficacy Stage occur in the same cycle (e.g. the dose of Talabostat in the Lead in Stage is 0.2 mg twice a day on days 1-7, followed a dose of 0.3 mg twice a day thereafter during the Efficacy Stage). In embodiments, Talabostat or a pharmaceutically acceptable salt thereof may be administered in divided doses during the Lead-in Stage at total daily dose of about 0.6 mg and during the Efficacy Stage a daily dose of about 0.4 mg. In embodiments, the patient is treated directly in the Efficacy Stage using a daily dose of about 0.4 mg or about 0.6 mg. In embodiments, the patient is treated directly in the Efficacy Stage using a total daily dose of about 0.6 mg. In embodiments, the patient is treated directly in the Efficacy Stage using a total daily dose of about 0.6 mg administered as a split dose 0.3 mg twice a day.

In embodiments, the Talabostat or a pharmaceutically acceptable salt thereof and Pembrolizumab are administered to a subject afflicted with one or more sarcoma selected from osteosarcoma and soft tissue sarcoma (for example, liposarcoma and myxoid sarcoma) in any desired number of treatment cycles as long as a clinical benefit is observed or until there is a complete response, confirmed progressive disease or unmanageable toxicity.

In embodiments, the daily dosages of Talabostat or a pharmaceutically acceptable salt thereof and Pembrolizumab used according to the disclosure is lower than the daily dosages of Talabostat or a pharmaceutically acceptable salt thereof or Pembrolizumab administered as a monotherapy to treat a subject afflicted with soft tissue sarcoma. In embodiments, the soft tissue sarcoma is selected from liposarcoma and myxoid sarcoma.

In embodiments, the combination therapy comprises administration of a 0.3 mg twice daily dose of Talabostat or a pharmaceutically acceptable salt thereof and 200 mg of pembrolizumab for at least 12 weeks (e.g. three 4-week cycles or four 3-week cycles) or at least 24 weeks (e.g. six 4-week cycles or eight 3-week cycles). In embodiments, the combination therapy is administered for at least 1-4 weeks after the patient achieves a complete response.

In embodiments, a single administration cycle is 21 days. In embodiments, Talabostat mesylate is administered once daily (QD) on days 1 to 14 of a 21-day cycle and 200 mg of pembrolizumab is administered intravenously (IV) on day 1 of every 21 day cycle. In embodiments, neither Talabostat nor Pembrolizumab are administered on days 15-21.

In embodiments, a single administration cycle is 21 days. In embodiments, Talabostat mesylate is administered twice daily at a dose of 0.3 mg on days 1 to 14 of a 21-day cycle and 200 mg of pembrolizumab is administered intravenously (IV) on day 1 of every 21 day cycle. In embodiments, Talabostat mesylate is administered at a dose of about 0.3 mg in the morning and about 0.3 mg in the evening on days 1 to 14 of a 21-day cycle and 200 mg of pembrolizumab is administered intravenously (IV) on Day 1 of every 21 day cycle. In embodiments, Talabostat mesylate is administered thrice daily at a dose of about 0.2 mg on days 1 to 14 of a 21-day cycle and 200 mg of pembrolizumab is administered intravenously (IV) on Day 1 of every 21 day cycle. In embodiments, Talabostat mesylate is administered at a dose of about 0.4 mg in the morning and about 0.2 mg in the evening on days 1 to 14 of a 21-day cycle and 200 mg pembrolizumab is administered intravenously (IV) on day 1 of every 21 day cycle. In embodiments, Talabostat mesylate is administered at a dose of about 0.2 mg in the morning and about 0.4 mg in the evening on days 1 to 14 of a 21-day cycle and 200 mg pembrolizumab is administered intravenously (IV) on day 1 of every 21 day cycle. In embodiments, Talabostat mesylate is administered twice daily at a dose of about 0.2 mg on days 1 to 7 and twice daily at a dose of about 0.3 mg on days 8 to 14 of a 21-day cycle and 200 mg of pembrolizumab is administered intravenously (IV) on day 1 of every 21 day cycle. In embodiments, Talabostat mesylate is administered twice daily at a dose of about 0.3 mg on days 1 to 3 and twice daily at a dose of about 0.3 mg on days 8 to 11 of a 21-day cycle and 200 mg of pembrolizumab is administered intravenously (IV) on day 1 of every 21 day cycle with a rest period from day 4-7 (no dose of Talabostat given).

In embodiments, a combination therapy of the disclosure is administered to a patient who has not been previously treated with a biotherapeutic or chemotherapeutic agent (i.e. is treatment-naïve). In embodiments, the combination therapy is administered to a patient who failed to achieve a sustained response after prior therapy with a biotherapeutic or chemotherapeutic agent (i.e. is treatment-experienced).

In embodiments, the subject was treated with a single daily 0.6 mg dose of Talabostat mesylate and experienced dose-limiting side effects.

A suitable time period of therapy can be determined by one skilled in the art (e.g., a physician). As can be appreciated in the art, a suitable period of time can be determined by one skilled in the art based on one or more of: the stage of disease in the patient, the mass and sex of the patient, clinical trial guidelines (e.g., those on the fda.gov website), and information on the approved drug label. For example a suitable time period of therapy can be, e.g., from 1 week to 2 years, 1 week to 22 months, 1 week to 20 months, 1 week to 18 months, 1 week to 16 months, 1 week to 14 months, 1 week to 12 months, 1 week to 10 months, 1 week to 8 months, 1 week to 6 months, 1 week to 4 months 1 week to 2 months, 1 week to 1 month, 2 weeks to 2 years, 2 weeks to 22 months, 2 weeks to 20 months, 2 weeks to 18 months, 2 weeks to 16 months, 2 weeks to 14 months, 2 weeks to 12 months, 2 weeks to 10 months, 2 weeks to 8 months, 2 weeks to 6 months, 2 weeks to 4 months, 2 weeks to 2 months, 2 weeks to 1 month, 1 month to 2 years, 1 month to 22 months, 1 month to 20 months, 1 month to 18 months, 1 month to 16 months, 1 month to 14 months, 1 month to 12 months, 1 month to 10 months, 1 month to 8 months, 1 month to 6 months, 1 month to 4 months, 1 month to 2 months, 2 months to 2 years, 2 months to 22 months, 2 months to 20 months, 2 months to 18 months, 2 months to 16 months, 2 months to 14 months, 2 months to 12 months, 2 months to 10 months, 2 months to 8 months, 2 months to 6 months, 2 months to 4 months, 3 months to 2 years, 3 months to 22 months, 3 months to 20 months, 3 months to 18 months, 3 months to 16 months, 3 months to 14 months, 3 months to 12 months, 3 months to 10 months, 3 months to 8 months, 3 months to 6 months, 4 months to 2 years, 4 months to 22 months, 4 months to 20 months, 4 months to 18 months, 4 months to 16 months, 4 months to 14 months, 4 months to 12 months, 4 months to 10 months, 4 months to 8 months, 4 months to 6 months, 6 months to 2 years, 6 months to 22 months, 6 months to 20 months, 6 months to 18 months, 6 months to 16 months, 6 months to 14 months, 6 months to 12 months, 6 months to 10 months, 6 months to 8 months, 8 months to 2 years, 8 months to 22 months, 8 months to 20 months, 8 months to 18 months, 8 months to 16 months, 8 months to 14 months, 8 months to 12 months, 8 months to 10 months, 10 months to 2 years, 10 months to 22 months, 10 months to 20 months, 10 months to 18 months, 10 months to 16 months, 10 months to 14 months, 10 months to 12 months, 12 months to 2 years, 12 months to 22 months, 12 months to 20 months, 12 months to 18 months, 12 months to 16 months, or 12 months to 14 months, inclusive.

### Pharmaceutical Formulations

In embodiments, the present disclosure provides pharmaceutical formulations comprising an effective amount of Talabostat or a pharmaceutically acceptable salt thereof and a therapeutically effective amount of Pembrolizumab together with one or more pharmaceutically acceptable carriers or adjuvants for treatment of sarcomas selected from osteosarcoma and soft tissue sarcoma (for example, liposarcoma and myxoid sarcoma). Any of the pharmaceutically acceptable carriers or adjuvants described herein, or known in the art, may be used. As used herein, the term “pharmaceutical formulation” refers to a formulation comprising Talabostat or a pharmaceutically acceptable salt thereof or a formulation comprising Pembrolizumab, wherein each formulation also comprises one or more pharmaceutically acceptable carriers or adjuvants. Pharmaceutically acceptable carriers or adjuvants are well-known to those skilled in the art, and usually depend on the chosen route of administration.

In embodiments, a first formulation comprises an effective amount of Talabostat or a pharmaceutically acceptable salt thereof and one or more pharmaceutically acceptable carriers or adjuvants and a second formulation comprises a therapeutically effective amount of Pembrolizumab and one or more pharmaceutically acceptable carriers or adjuvants are administered to produce a synergistic effect in treating a subject afflicted with one or more sarcomas (e.g. soft tissue sarcoma). In embodiments, the soft tissue sarcoma is liposarcoma or myxoid sarcoma.

The pharmaceutical formulations may be formulated in a variety of ways, including for example, liquid, semi-solid and solid dosage forms, such as liquid solutions (e.g., injectable and infusible solutions), dispersions or suspensions, tablets, pills, powders, liposomes and suppositories. In embodiments, the compositions may be formulated as the injectable or infusible solutions. In embodiments, the formulation is in a form suitable for oral, intravenous, intraarterial, intramuscular, subcutaneous, parenteral, transmucosal, transdermal, or topical administration. In embodiments, the formulation may be formulated as an immediate, controlled, extended or delayed release composition.

In embodiments, the formulation comprising Talabostat or a pharmaceutically acceptable salt thereof may be administered orally. In embodiments, the formulation comprising Pembrolizumab may be administered parenterally. As used herein, the term “parenteral” includes subcutaneous, intravenous, intra-arterial, intraperitoneal, intracardiac, intrathecal, and intramuscular injection, as well as infusion injections.

In embodiments, liquid pharmaceutical formulations for parenteral administration may be formulated for administration by injection or continuous infusion. In embodiments, parenteral formulations can include: prefilled syringes, vials, powder for infusion, for reconstitution, concentrate for infusion to be diluted before delivery (ready to dilute), solutions (ready to use).

In embodiments, injectable pharmaceutical formulations can be aqueous isotonic solutions or suspensions. In embodiments, suppositories can be prepared from fatty emulsions or suspensions.

In embodiments, pharmaceutical formulations formulated for parenteral administration (e.g. via intravenous injection) may conveniently include a liquid carrier, or may be reconstituted into a liquid solution or suspension for parenteral administration.

In general, such formulations typically comprise a pharmaceutically acceptable carrier or adjuvant. As used herein, the term “pharmaceutically acceptable” means approved by a government regulatory agency or listed in the U.S. Pharmacopoeia or another generally recognized pharmacopoeia for use in animals, particularly in humans.

Pharmaceutical formulations of Pembrolizumab for intravenous administration may be purchased from the marketplace or prepared using conventional formulation techniques. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride, hexamethonium chloride, benzalkonium chloride, benzethonium chloride, phenol, butyl or benzyl alcohol, chlorobutanol, thimerosal, alkyl parabens such as methyl or propyl paraben, catechol, resorcinol, cyclohexanol, 3-pentanol and m-cresol; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; chelating agents such as EDTA; monosaccharides, disaccharides, and other carbohydrates including sugars such as sucrose, mannitol, trehalose or sorbitol, glucose, mannose, or dextrins; salt-forming counter-ions such as sodium; metal complexes (for example, Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). The carrier can be a solvent or reconstitution medium or dispersion medium containing, for example, water, ethanol, polyol (for example, glycerol, propylene glycol, and liquid polyethylene glycol, and the like), and suitable mixtures thereof. The pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringeability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, liquid polyethylene glycol, or the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

In embodiments, the formulation includes isotonic agents, for example, sugars, polyalcohols, such as mannitol, sorbitol, or sodium chloride. For intravenous administration, suitable carriers include physiological saline, bacteriostatic water, Cremophor EL (BASF, Parsippany, N.J.) or phosphate buffered saline (PBS). Prolonged absorption of the injectable compositions can be brought about by including in the composition an agent which delays absorption, for example, aluminum monostearate and gelatin.

Sterile injectable solutions can be prepared by incorporating the molecule, by itself or in combination with other active agents, in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, one method of preparation is vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Such articles of manufacture will preferably have labels or package inserts indicating that the associated compositions are useful for treating a subject suffering from or predisposed to autoimmune or neoplastic disorders. The pharmaceutical formulations may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

Pharmaceutical formulations of Talabostat or a pharmaceutically acceptable salt thereof for oral use herein may be administered, for example, in the form of tablets, capsules, powders, dispersible granules, sachets etc., or as aqueous solutions or suspensions, preferably tablets. Oral compositions generally include an inert carrier (for example, diluent) or an edible carrier. The formulations may be enclosed in a gelatin capsule or compressed into a tablet. Pharmaceutically compatible binding agents, and/or adjuvant materials can be included as part of the formulation. The tablets, pills, capsules, granules, sachets, troches and the like can contain any of the following ingredients, or compounds of a similar nature; a binder such as microcrystalline cellulose, gum tragacanth or gelatin; an excipient such as starch or lactose, a disintegrating agent such as alginic acid, primogel, or corn starch; a lubricant such as magnesium stearate or stearates; a glidant such as colloidal silicon dioxide; a sweetening agent such as sucrose or saccharin; or a flavoring agent such as peppermint, methyl salicylate, or orange flavoring.

In embodiments, an oral pharmaceutical formulation comprising an effective amount Talabostat or a pharmaceutically acceptable salt thereof described herein may comprise one or more pharmaceutically acceptable carriers or adjuvants selected from the group comprising a bulking agent, buffer, surfactant, and pH modifier. The pharmaceutical formulation may be adjusted to give an appropriate pH.

In embodiments, Talabostat or a pharmaceutically acceptable salt thereof is formulated as a tablet for oral administration. The pharmaceutical tablet may be an immediate release or a modified release tablet. Tablet may be in the form of matrix or coated form.

In embodiments, the various processes of making above mentioned formulations or compositions are included and such formulations can be manufactured by any of the processes known in the art.

An exemplary immediate release tablet comprises an effective amount of Talabostat or a pharmaceutically acceptable salt thereof and one or more pharmaceutically acceptable carriers selected from diluents, binders, disintegrants, glidants, lubricants, pH modifying agents and combinations thereof.

Diluents: one or more diluents comprise, but are not limited to dibasic calcium phosphate, pullulan, maltodextrin, isomalt, sugar pellets, mannitol, spray-dried mannitol, microcrystalline cellulose, dibasic calcium phosphate dihydrate, lactose, sugars, sorbitol, mixture of microcrystalline cellulose and guar gum (AVICEL® CE-15), mixture of mannitol, polyplasdone and syloid (Pharmaburst), mixture of mannitol, crospovidone and polyvinyl acetate (LUDIFLASH®), isomalt, PANEXCEA® (MCC USP, crospovidone USP and hydroxypropylmethylcellulose), F-MELT®, sucrose, calcium salts and similar inorganic salts, heavy magnesium carbonate and the like, and the mixtures thereof. Preferably, it is lactose or microcrystalline cellulose.

Binders: one or more binders comprise, but are not limited to, low-substituted hydroxypropyl cellulose, xanthan gum, polyvinylpyrrolidone (povidone), gelatin, sugars, glucose, natural gums, gums, synthetic celluloses, polymethacrylate, hydroxypropyl methylcellulose, hydroxypropyl cellulose, carboxymethyl cellulose, methyl cellulose, and other cellulose derivatives and the like, and the mixtures thereof. Preferably, the binder is polyvinylpyrrolidone or hydroxypropyl cellulose or hydroxypropyl methylcellulose.

Disintegrants: one or more binders comprise, but are not limited to, at least one or a mixture of sodium starch glycolate, croscarmellose sodium, crospovidone, sodium alginate, gums, starch, and magnesium aluminum silicate. Preferably, the disintegrant is sodium starch glycolate.

Lubricants: one or lubricants comprise, but are not limited to sodium stearyl fumarate, sodium lauryl sulphate, magnesium stearate, polyethylene glycol, metal stearates, hydrogenated castor oil and the like, and the mixtures thereof. Preferably, the lubricant is magnesium stearate.

Glidants: one or glidants comprise, but are not limited to, stearic acid, colloidal silicon dioxide, talc, aluminum silicate and the like, and the mixtures thereof. Preferably, it is talc.

pH modifying agents: one or more pH modifying agents comprise, but are not limited to organic acid or its salts like phosphoric acid, citric acid and the like.

In embodiments, the relative percentages of the ingredients in tablet formulations of Talabostat is given below in Table 1:

TABLE 1

| | Amount |
|---|---|
| Formulation Content | (w/w %) |
| Talabostat (as Talabostat | 0.01-2 |
| mesylate) | |
| Binder | 1-50 |
| Disintegrant | 1-15 |
| Lubricant | 0.1-5 |
| Diluent | 30-98 |
| pH modifying agent | 0-15 |

An exemplary immediate release tablet of Talabostat mesylate is given below in Table 2:

TABLE 2

| | Preferred ranges | Amount |
|---|---|---|
| Formulation content | (w/w %) | (w/w %) |
| Talabostat mesylate (69% free | 0.01-2 | 0.145 |
| base) | | |
| Polyvinylpyrrolidone or | 1-50 | 1.00 |
| hydroxypropylcellulose or | | |
| hydroxypropylmethylcellulose | | |
| or pregelatinized starch as a | | |
| binder | | |
| Sodium starch glycolate or | 1-15 | 2.5 |
| crospovidone as a disintegrant | | |
| Stearic acid as a lubricant | 0.1-5 | 1.5 |
| Lactose as a diluent | 30-90 | 85.315 |
| Microcrystalline cellulose as a | 5-20 | 9.480 |
| diluent | | |
| Sodium phosphate monobasic, | 0-15 | 0.060 |
| monohydrate as a pH | | |
| modifying agent | | |
| Phosphoric acid as a pH | For pH adjustment | For pH adjustment |
| modifying agent | | |

In embodiments, Talabostat or a pharmaceutically acceptable salt thereof may be formulated as a modified release matrix tablet. An exemplary extended release tablet comprises an effective amount of Talabostat or a pharmaceutically acceptable salt thereof and pharmaceutically-acceptable carriers or adjuvants are selected from diluents, binders, modified release material, glidants, lubricants, colorants and combinations thereof. Alternatively, a modified release tablet comprises immediate release core and coating wherein said coating comprises modified release material and other pharmaceutical excipients.

Modified release materials comprise, but are not limited to: polyvinylpyrrolidone (K90), Hydroxypropylmethylcellulose (K4M, K10), hydroxypropylcellulose (high viscosity grade), carnauba wax, glyceryl behenate, castor wax, polyvinyl acetate, carboxymethyl ethyl cellulose, ethylcellulose, cellulose phthalates or succinates, in particular cellulose acetate phthalate and hydroxypropylmethylcellulose phthalate, hydroxypropylmethylcellulose succinate or hydroxypropylmethylcellulose acetate succinate; high molecular polyalkylene oxides such as polyethylene oxide and polypropylene oxide and copolymers of ethylene oxide and propylene oxide and the like. Particular modified release materials include polyvinylpyrrolidone (K90), hydroxypropylmethylcellulose (K4M, K10), hydroxypropylcellulose (high viscosity grade-HF), polyethylene oxide and the like. A modified release material may conveniently be present in the range of 10-50% wt. of the tablet.

An exemplary modified release tablet of Talabostat or a pharmaceutically acceptable salt thereof is given below in Table 3:

TABLE 3

| | Amount |
|---|---|
| Formulation content | (w/w %) |
| Talabostat as an API (available as | 0.01-2 |
| Talabostat mesylate) | |
| Polyvinylpyrrolidone (K90) or | 10-50 |
| hydroxypropylmethylcellulose (K4M, | |
| K10) or hydroxypropylcellulose (high | |
| viscosity grade-HF) or polyethylene | |
| oxide as a modified release material | |
| Sodium starch glycolate or crospovidone | 0-10 |
| as a disintegrant | |
| Magnesium stearate or stearic acid | 0.1-10 |
| as a lubricant | |
| Citric acid or phosphoric acid as | 0-15 |
| a pH modifying agent | |
| Lactose as a filler | 30-90 |

In embodiments, the amount of Talabostat in a unit dose is about 50 micrograms per tablet, about 100 micrograms per tablet, about 200 micrograms per tablet, about 300 micrograms per tablet, about 400 micrograms per tablet, about 500 micrograms per tablet, about 600 micrograms per tablet, about 700 micrograms per tablet, about 800 micrograms per tablet.

Various methods can be used for manufacturing tablets of Talabostat or a pharmaceutically acceptable salt thereof for use in the treatment regimen of this disclosure. One process includes dissolving Talabostat in a suitable solvent (with or without binder) and this solution is distributed uniformly over filler particles (which may contain other materials) to form agglomerated particles/granules. Wet granulation, coating or spraying processes can also be used. Granules may be appropriately sized or may be further processed by a dry granulation/slugging/roller compaction method followed by a milling step to achieve suitable granules of specific particle size distribution. The sized granules may be further blended with other components and/or and then lubricated in a suitable blender and compressed into tablets of specific dimensions using appropriate tooling. Coating of the tablets, where appropriate, may be performed using conventional methods and standard equipment.

### Kits

In embodiments, the kit includes a formulation comprising Talabostat or pharmaceutically acceptable salt thereof and a formulation comprising Pembrolizumab with or without instructions for their use. The combined therapeutics can be manufactured and/or formulated by the same or different manufacturers. The combination therapeutics may thus be entirely separate pharmaceutical dosage forms or pharmaceutical compositions that are also sold independently of each other. In embodiments, instructions for their combined use are provided: (i) prior to release to physicians (e.g. in the case of a “kit of part” comprising a first therapeutic agent and the other therapeutic agent); (ii) by the physicians themselves (or under the guidance of a physician) shortly before administration; (iii) the patient themselves by a physician or medical staff.

In embodiments, a single bolus dose may be administered. In embodiments, several divided doses may be administered over time. In embodiments a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of active compound calculated to produce a desired therapeutic effect. In embodiments, the dosage unit forms of the disclosure are dictated by and directly dependent on the unique characteristics of the active compound and the particular therapeutic or prophylactic effect to be achieved.

In embodiments, the kit comprises a package insert comprising instructions for using Talabostat or pharmaceutically acceptable salt thereof and Pembrolizumab to treat or delay progression of cancer in a subject or to enhance immune function of a subject having cancer. In embodiments, the kit comprises an effective amount of Talabostat or pharmaceutically acceptable salt thereof and a therapeutically effective amount of Pembrolizumab and package insert comprising instructions for using the same to treat or delay progression of cancer in a subject or to enhance immune function of a subject having cancer. In embodiments, the kit comprises Talabostat or pharmaceutically acceptable salt thereof and Pembrolizumab, and a package insert comprising instructions for using the same to treat or delay progression of sarcoma in a subject or to enhance immune function of a subject having sarcoma selected from soft tissue sarcoma. In embodiments, the soft tissue sarcoma is selected from liposarcoma and myxoid sarcoma.

In embodiments, the kit comprises a container that includes, but is not limited to bottles, vials (e.g., dual chamber vials), syringes (such as single or dual chamber syringes) and test tubes. The container may be formed from a variety of materials such as glass or plastic. In embodiments, the kit may comprise a label (e.g., on or associated with the container) or a package insert. The label or the package insert may indicate that the compound contained therein may be useful or intended for treating or delaying progression of cancer in a subject or for enhancing immune function of a subject having cancer selected from soft tissue sarcoma, liposarcoma and myxoid sarcoma.

The kit may further comprise other materials desirable from a commercial and user standpoint, including other buffers, diluents, filters, needles, and syringes.

### Outcomes

Patients afflicted with one or more advanced sarcomas selected from osteosarcoma and soft tissue sarcoma administered a 0.3 mg twice daily dose of Talabostat or a pharmaceutically acceptable salt thereof and a therapeutically effective amount of Pembrolizumab according to the treatment regimen disclosed herein preferably experience improvement in at least one sign of cancer. In embodiments, improvement may be measured by a reduction in the quantity and/or size of measurable tumor lesions. In embodiments, the soft tissue sarcoma is liposarcoma. In embodiments, the liposarcoma is myxoid sarcoma. In embodiments, lesions can be measured using X-rays or CT or MRI scans. In embodiments, cytology or histology can be used to evaluate responsiveness to the therapy. In embodiments, extension of progression free survival and/or overall survival may be provided to a patient afflicted with sarcoma such as soft tissue sarcoma.

In embodiments, the anti-tumor response is a tumor specific response, a clinical response, a decrease in tumor size/volume, a decrease in tumor specific biomarkers, increase in anti-tumor cytokines or a combination thereof.

In embodiments, the clinical response is a decreased tumor growth and/or a decrease in tumor size. In embodiments, the initiating, sustaining or enhancing an anti-tumor immune response is for the treatment of soft tissue sarcoma. In embodiments, the soft tissue sarcoma is liposarcoma. In embodiments, the liposarcoma is myxoid sarcoma.

In embodiments, the anti-tumor response is inhibiting tumor growth, inducing tumor cell death, tumor regression, preventing or delaying tumor recurrence, tumor growth, tumor spread or tumor elimination.

In embodiments, the anti-tumor response is reduction in metastasis, delay in metastasis or prevention of metastasis. In embodiments, the anti-tumor response is prevention of metastasis.

In embodiments, the tumor response is a decrease in the number of tumor cells. In embodiments, the tumor response is a decreased rate in tumor growth. In embodiments, the tumor response is a block in the dipeptidyl peptidase enzyme activity. In embodiments, the tumor response is an induction of proinflammatory cytokine response and a cytotoxic T cell response.

The treatment may result in an inhibition of tumor size more than about 10%, more than about 20%, more than about 21%, more than about 22%, more than about 23%, more than about 24%, more than about 25%, more than about 26%, more than about 27%, more than about 28%, more than about 29%, more than about 30%, more than about 31%, more than about 32%, more than about 33%, more than about 34%, more than about 35%, more than about 36%, more than about 37%, more than about 38%, more than about 39%, more than about 40%, more than about 41%, more than about 42%, more than about 43%, more than about 44%, more than about 45%, more than about 46%, more than about 47%, more than about 48%, more than about 49%, more than about 50%, more than about 51%, more than about 52%, more than about 53%, more than about 54%, more than about 55%, more than about 56%, more than about 57%, more than about 58%, more than about 59%, more than about 60%, more than about 61%, more than about 62%, more than about 63%, more than about 64%, more than about 65%, more than 66%, more than 67%, more than about 68%, more than about 69%, more than about 70%, more than about 71%, more than about 72%, more than about 73%, more than about 74%, more than about 75%, more than about 76%, more than about 77%, more than about 78%, more than about 79%, more than about 80%, more than about 81%, more than about 82%, more than about 83%, more than about 84%, more than about 85%, more than about 86%, more than about 87%, more than about 88%, more than about 89%, more than about 90%, more than about 91%, more than about 92%, more than about 93%, more than about 94%, more than about 95%, more than about 96%, more than about 97%, more than about 98%, more than about 99% up to about 100%.

In embodiments, the methods provided herein can result in a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 95%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the volume of one or more sarcomas selected from soft tissue sarcoma (such as liposarcoma or myxoid sarcoma) or osteosarcoma in a patient following treatment with the combination therapy for a period of time between 1 day and 2 years (e.g., between 1 day and 22 months, between 1 day and 20 months, between 1 day and 18 months, between 1 day and 16 months, between 1 day and 14 months, between 1 day and 12 months, between 1 day and 10 months, between 1 day and 9 months, between 1 day and 8 months, between 1 day and 7 months, between 1 day and 6 months, between 1 day and 5 months, between 1 day and 4 months, between 1 day and 3 months, between 1 day and 2 months, between 1 day and 1 month, between one week and 2 years, between 1 week and 22 months, between 1 week and 20 months, between 1 week and 18 months, between 1 week and 16 months, between 1 week and 14 months, between 1 week and 12 months, between 1 week and 10 months, between 1 week and 9 months, between 1 week and 8 months, between 1 week and 7 months, between 1 week and 6 months, between 1 week and 5 months, between 1 week and 4 months, between 1 week and 3 months, between 1 week and 2 months, between 1 week and 1 month, between 2 weeks and 2 years, between 2 weeks and 22 months, between 2 weeks and 20 months, between 2 weeks and 18 months, between 2 weeks and 16 months, between 2 weeks and 14 months, between 2 weeks and 12 months, between 2 weeks and 10 months, between 2 weeks and 9 months, between 2 weeks and 8 months, between 2 weeks and 7 months, between 2 weeks and 6 months, between 2 weeks and 5 months, between 2 weeks and 4 months, between 2 weeks and 3 months, between 2 weeks and 2 months, between 2 weeks and 1 month, between 1 month and 2 years, between 1 month and 22 months, between 1 month and 20 months, between 1 month and 18 months, between 1 month and 16 months, between 1 month and 14 months, between 1 month and 12 months, between 1 month and 10 months, between 1 month and 9 months, between 1 month and 8 months, between 1 month and 7 months, between 1 month and 6 months, between 1 month and 6 months, between 1 month and 5 months, between 1 month and 4 months, between 1 month and 3 months, between 1 month and 2 months, between 2 months and 2 years, between 2 months and 22 months, between 2 months and 20 months, between 2 months and 18 months, between 2 months and 16 months, between 2 months and 14 months, between 2 months and 12 months, between 2 months and 10 months, between 2 months and 9 months, between 2 months and 8 months, between 2 months and 7 months, between 2 months and 6 months, or between 2 months and 5 months, between 2 months and 4 months, between 3 months and 2 years, between 3 months and 22 months, between 3 months and 20 months, between 3 months and 18 months, between 3 months and 16 months, between 3 months and 14 months, between 3 months and 12 months, between 3 months and 10 months, between 3 months and 8 months, between 3 months and 6 months, between 4 months and 2 years, between 4 months and 22 months, between 4 months and 20 months, between 4 months and 18 months, between 4 months and 16 months, between 4 months and 14 months, between 4 months and 12 months, between 4 months and 10 months, between 4 months and 8 months, between 4 months and 6 months, between 6 months and 2 years, between 6 months and 22 months, between 6 months and 20 months, between 6 months and 18 months, between 6 months and 16 months, between 6 months and 14 months, between 6 months and 12 months, between 6 months and 10 months, or between 6 months and 8 months) (e.g., as compared to the size of said one or more solid tumors in the patient prior to treatment).

In embodiments, the methods provided herein can provide for 1% to 99% (e.g. 1% to 98%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 95%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the risk of developing a metastasis or the risk of developing an additional metastasis in a patient having soft tissue sarcoma. In embodiments, the soft tissue sarcoma is liposarcoma. In embodiments, the liposarcoma is myxoid sarcoma. In embodiments, the methods described herein can result in an increase (e.g., a 1% to 400%, 1% to 380%, 1% to 360%, 1% to 340%, 1% to 320%, 1% to 300%, 1% to 280%, 1% to 260%, 1% to 240%, 1% to 220%, 1% to 200%, 1% to 180%, 1% to 160%, 1% to 140%, 1% to 120%, 1% to 100%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 400%, 5% to 380%, 5% to 360%, 5% to 340%, 5% to 320%, 5% to 300%, 5% to 280%, 5% to 260%, 5% to 240%, 5% to 220%, 5% to 200%, 5% to 180%, 5% to 160%, 5% to 140%, 5% to 120%, 5% to 100%, 5% to 90%, 5% to 80%, 5% to 70%, 5% to 60%, 5% to 50%, 5% to 40%, 5% to 30%, 5% to 20%, 5% to 10%, 10% to 400%, 10% to 380%, 10% to 360%, 10% to 340%, 10% to 320%, 10% to 300%, 10% to 280%, 10% to 260%, 10% to 240%, 10% to 220%, 10% to 200%, 10% to 180%, 10% to 160%, 10% to 140%, 10% to 120%, 10% to 100%, 10% to 90%, 10% to 80%, 10% to 70%, 10% to 60%, 10% to 50%, 10% to 40%, 10% to 30%, 10% to 20%, 20% to 400%, 20% to 380%, 20% to 360%, 20% to 340%, 20% to 320%, 20% to 300%, 20% to 280%, 20% to 260%, 20% to 240%, 20% to 220%, 20% to 200%, 20% to 180%, 20% to 160%, 20% to 140%, 20% to 120%, 20% to 100%, 20% to 90%, 20% to 80%, 20% to 70%, 20% to 60%, 20% to 50%, 20% to 40%, 20% to 30%, 30% to 400%, 30% to 380%, 30% to 360%, 30% to 340%, 30% to 320%, 30% to 300%, 30% to 280%, 30% to 260%, 30% to 240%, 30% to 220%, 30% to 200%, 30% to 180%, 30% to 160%, 30% to 140%, 30% to 120%, 30% to 100%, 30% to 90%, 30% to 80%, 30% to 70%, 30% to 60%, 30% to 50%, 30% to 40%, 40% to 400%, 40% to 380%, 40% to 360%, 40% to 340%, 40% to 320%, 40% to 300%, 40% to 280%, 40% to 260%, 40% to 240%, 40% to 220%, 40% to 200%, 40% to 180%, 40% to 160%, 40% to 140%, 40% to 120%, 40% to 100%, 40% to 90%, 40% to 80%, 40% to 70%, 40% to 60%, 40% to 50%, 50% to 400%, 50% to 380%, 50% to 360%, 50% to 340%, 50% to 320%, 50% to 300%, 50% to 280%, 50% to 260%, 50% to 240%, 50% to 220%, 50% to 200%, 50% to 180%, 50% to 160%, 50% to 140%, 50% to 140%, 50% to 120%, 50% to 100%, 50% to 90%, 50% to 80%, 50% to 70%, 50% to 60%, 60% to 400%, 60% to 380%, 60% to 360%, 60% to 340%, 60% to 320%, 60% to 300%, 60% to 280%, 60% to 260%, 60% to 240%, 60% to 220%, 60% to 200%, 60% to 180%, 60% to 160%, 60% to 140%, 60% to 120%, 60% to 100%, 60% to 90%, 60% to 80%, 60% to 70%, 70% to 400%, 70% to 380%, 70% to 360%, 70% to 340%, 70% to 320%, 70% to 300%, 70% to 280%, 70% to 260%, 70% to 240%, 70% to 220%, 70% to 200%, 70% to 180%, 70% to 160%, 70% to 140%, 70% to 120%, to 100%, 70% to 90%, 70% to 80%, 80% to 400%, 80% to 380%, 80% to 360%, 80% to 340%, 80% to 320%, 80% to 300%, 80% to 280%, 80% to 260%, 80% to 240%, 80% to 220%, 80% to 200%, 80% to 180%, 80% to 160%, 80% to 140%, 80% to 120%, 80% to 100%, 80% to 90%, 90% to 400%, 90% to 380%, 90% to 360%, 90% to 340%, 90% to 320%, 90% to 300%, 90% to 280%, 90% to 260%, 90% to 240%, 90% to 220%, 90% to 200%, 90% to 180%, 90% to 160%, 90% to 140%, 90% to 120%, 90% to 100%, 100% to 400%, 100% to 380%, 100% to 360%, 100% to 340%, 100% to 320%, 100% to 300%, 100% to 280%, 100% to 260%, 100% to 240%, 100% to 220%, 100% to 200%, 100% to 180%, 100% to 160%, 100% to 140%, 100% to 120%, 120% to 400%, 120% to 380%, 120% to 360%, 120% to 340%, 120% to 320%, 120% to 300%, 120% to 280%, 120% to 260%, 120% to 240%, 120% to 220%, 120% to 200%, 120% to 180%, 120% to 160%, 120% to 140%, 140% to 400%, 140% to 380%, 140% to 360%, 140% to 340%, 140% to 320%, 140% to 300%, 140% to 280%, 140% to 260%, 140% to 240%, 140% to 220%, 140% to 200%, 140% to 180%, 140% to 160%, 160% to 400%, 160% to 380%, 160% to 360%, 160% to 340%, 160% to 320%, 160% to 300%, 160% to 280%, 160% to 260%, 160% to 240%, 160% to 220%, 160% to 200%, 160% to 180%, 180% to 400%, 180% to 380%, 180% to 360%, 180% to 340%, 180% to 320%, 180% to 300%, 180% to 280%, 180% to 260%, 180% to 240%, 180% to 220%, 180% to 200%, 200% to 400%, 200% to 380%, 200% to 360%, 200% to 340%, 200% to 320%, 200% to 300%, 200% to 280%, 200% to 260%, 200% to 240%, 200% to 220%, 220% to 400%, 220% to 380%, 220% to 360%, 220% to 340%, 220% to 320%, 220% to 300%, 220% to 280%, 220% to 260%, 220% to 240%, 240% to 400%, 240% to 380%, 240% to 360%, 240% to 340%, 240% to 320%, 240% to 300%, 240% to 280%, 240% to 260%, 260% to 400%, 260% to 380%, 260% to 360%, 260% to 340%, 260% to 320%, 260% to 300%, 260% to 280%, 280% to 400%, 280% to 380%, 280% to 360%, 280% to 340%, 280% to 320%, 280% to 300%, 300% to 400%, 300% to 380%, 300% to 360%, 300% to 340%, or 300% to 320%) in the time of survival of the patient (e.g., as compared to a patient having a similar cancer and administered a different treatment or not receiving a treatment).

In embodiments, patients afflicted with soft tissue sarcoma administered a 0.3 mg twice daily dose of Talabostat or a pharmaceutically acceptable salt thereof on one or more days of a treatment cycle, and a therapeutically effective amount of Pembrolizumab may exhibit a complete response (CR), a partial response (PR), stable disease (SD), immune-related complete disease (irCR), immune-related partial response (irPR), or immune-related stable disease (irSD). In embodiments, patients afflicted with liposarcoma administered Talabostat or a pharmaceutically acceptable salt thereof and Pembrolizumab may exhibit a complete response (CR), a partial response (PR), stable disease (SD), immune-related complete disease (irCR), immune-related partial response (irPR), or immune-related stable disease (irSD). In embodiment, patients afflicted with myxoid sarcoma administered Talabostat or a pharmaceutically acceptable salt thereof and Pembrolizumab may exhibit a complete response (CR), a partial response (PR), stable disease (SD), immune-related complete disease (irCR), immune-related partial response (irPR), or immune-related stable disease (irSD).

In embodiments, the treatment results in a complete response (CR), a partial response (PR), stable disease (SD), immune-related complete disease (irCR), immune-related partial response (irPR), or immune-related stable disease (irSD). In embodiments, the treatment results in a complete response (CR). In embodiments, the treatment results in a partial response (PR). In embodiments, the treatment results in stable disease (SD). In embodiments, the treatment results in immune-related complete disease (irCR). In embodiments, the treatment results in immune-related partial response (irPR). In embodiments, the treatment results in immune-related stable disease (irSD).

The term “complete response” as used herein refers to disappearance of all target lesions. Any pathological lymph nodes (whether target or nontarget) must have reduction in short axis to <10 mm.

The term “partial response” as used herein refers to at least a 30% decrease in the sum of diameters of target lesions, taking as reference the baseline sum diameters.

The term “stable disease” as used herein refers to neither sufficient shrinkage from the baseline study to qualify for PR nor sufficient increase to qualify for PD, taking as reference the smallest sum diameters while on study. The term “progressive disease” as used herein refers to at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of 1 or more new lesions is also considered progression).

In embodiment, the patients afflicted with soft tissue sarcoma administered according to the treatment regimen achieves a complete or partial disease response or better, as measured by RECIST 1.1.

In embodiment, the patients afflicted with liposarcoma administered according to the treatment regimen achieves a complete response or partial response or better, as measured by RECIST 1.1.

In embodiments, the subject experiences less treatment-related adverse events (TRAEs) relative to a subject with same cancer administered a single 0.6 mg daily dose of Talabostat. In embodiments, TRAEs include, but are not limited to hypotension, dizziness, headache, syncope, dyspnea, chills, pyrexia, malaise, weakness, edema/peripheral swelling, hypovolemia, hypothermia, fatigue, nausea, vomiting, diaphoresis, flushing, migraine, diarrhea, constipation, alopecia, pharyngitis, chest pain, anorexia, weight increase, weight decrease, vertigo, syncope, conjunctivitis, blurred vision, pallor, pruritus, rash, fungal vaginosis, hyperglycemia, hyperkalemia, hypokalemia, hoarseness, dyspnea, anoxia, deep venous thrombosis, upper respiratory infection, blood in stool, dizziness, rigors, sepsis, pain, hypereosinophilia, dehydration, electrolyte imbalance, arthralgia, rhabdomyolysis, myalgia, constipation, hypocalcemia, neutropenia, febrile neutropenia, anemia, leukopenia, pancytopenia, lymphopenia, somnolence, insomnia, epistaxis, dyspepsia, dysgeusia, thrombocytopenia, cyanosis peripheral, hypovolemic shock, respiratory failure, cough, pneumonitis, cardiac tamponade, acidosis, renal failure and cardiac arrest. In embodiments, the subject experiences no TRAEs.

In embodiments, the subject experiences an increase in pro-inflammatory cytokines relative to a subject that is administered a single 0.6 mg daily dose of Talabostat. In embodiments, pro-inflammatory cytokines include, but are not limited to IL-6, IL-8, IL-18, IFN-γ, and IL-1β. In embodiments, the pro-inflammatory cytokines are one or more of IL-18 and IFN-γ.

In embodiments, patients afflicted with soft tissue sarcoma administered a 0.3 mg twice daily dose of Talabostat or a pharmaceutically acceptable salt thereof on one or more days of a treatment cycle, and a therapeutically effective amount of Pembrolizumab on day 1 of the treatment cycle may experience tumor shrinkage and/or decrease in growth rate, i.e., suppression of tumor growth. In embodiments, unwanted cell proliferation may be reduced or inhibited.

Soft tissue sarcoma is a type of cancer that develops from the soft tissues of the body, such as fat, muscle, nerves, fibrous tissues, blood vessels or deep skin tissues. They usually develop in the arms or legs but can occur anywhere in the body, including the trunk, head, neck, internal organs, and the area in the back of the abdomen. The term “advanced sarcoma” refers to the sarcoma that involves or attaches to nearby tissues or organs wherein such carcinoma is not curable by local modalities of treatment, such as surgery or radiotherapy. “Metastatic sarcoma” is a sarcoma which has spread from the part of the body where it started (the primary site) to other parts of the body through blood stream or lymph node. Previously treated sarcoma may be defined as the sarcoma which has been previously treated with one or more regimens of systemic therapies for sarcoma, however the sarcomas either relapsed or progressed.

In embodiments, patients afflicted with soft tissue sarcoma may experience one or more of the following after administration of Talabostat and Pembrolizumab: the number of cancer cells may be reduced; tumor size may be reduced; cancer cell in filtration into peripheral organs may be inhibited, retarded, slowed, or stopped; tumor metastasis may be slowed or inhibited; tumor growth may be inhibited; recurrence of tumor may be prevented or delayed; one or more of the symptoms associated with cancer may be alleviated.

In embodiments, the patient afflicted with one or more soft tissue sarcoma administered a 0.3 mg twice daily dose of Talabostat or a pharmaceutically acceptable salt thereof on one or more days of a treatment cycle and about 200 mg of Pembrolizumab on day 1 of the treatment cycle may exhibit at least one therapeutic effect selected from the group consisting of reduction in size of a tumor, reduction in number of metastatic lesions appearing over time, complete remission, partial remission, or stable disease.

In embodiments, the treatment regimen produces a clinical benefit rate (CBR=CR+PR+SD; e.g. ≥6 months) better than that achieved by a monotherapy of Talabostat or a pharmaceutically acceptable salt thereof, or Pembrolizumab.

In embodiments, the improvement of clinical benefit rate achieved may be about 20%, 30%, 40%, 50%, 60%, 70%, 80% or more compared to a monotherapy with Talabostat or Pembrolizumab.

In embodiments, the treatment results in the CD8+ T cells in the subject having enhanced priming, activation, proliferation and/or cytolytic activity when compared to a monotherapy with Talabostat or Pembrolizumab.

In embodiments, the number of CD4+ and/or CD8+ T cells is elevated relative to prior to administration of the combination.

In embodiments, the activated CD4+ and/or CD8+ T cells is characterized by γ-IFN+ producing CD4+ and/or CD8+ T cells and/or enhanced cytolytic activity relative to prior to the administration of the combination.

In embodiments, the CD4+ and/or CD8+ T cells exhibit increased release of cytokines selected from the group consisting of G-CSF, MCP-1, Eotaxin, IFN-γ, KC, TNF-α and interleukins (IL-5, IL-6, IL-1β, IL-12p70, IL 18).

In embodiments, the CD4+ and/or CD8+ T cell is an effector memory T cell. In embodiments, the CD4+ and/or CD8+ effector memory T cell is characterized by γ-IFN+ producing CD4+ and/or CD8+ T cells and/or enhanced cytolytic activity.

In embodiments, the serum levels of cytokine IL-18 and/or chemokine GM-CSF, G-CSF in the subject are increased in the presence of combination of Talabostat or a pharmaceutically acceptable salt thereof and Pembrolizumab as compared to a monotherapy with Talabostat or Pembrolizumab.

In embodiments, the cancer has elevated levels of T-cell infiltration when a combination of Talabostat or a pharmaceutically acceptable salt thereof, and Pembrolizumab is used compared to a monotherapy with Talabostat or Pembrolizumab.

In embodiments, the cancer has suppressed/decreased levels of T-regulatory cells in the presence of a combination of Talabostat or a pharmaceutically acceptable salt thereof and Pembrolizumab compared to a monotherapy with Talabostat or Pembrolizumab. In embodiments, the cancer has increased levels of NK cells and macrophages in the presence of combination of Talabostat or a pharmaceutically acceptable salt thereof and Pembrolizumab when used according to the treatment regimen described herein, when compared to a monotherapy with Talabostat or Pembrolizumab.

With respect to target lesions, responses to the treatment regimen described herein may include: Complete response (CR), Partial Response (PR), Progressive Disease (PD), Stable Disease (SD), Immune-related Complete Response (irCR), Immune-related Partial Response (irPR), Immune-related Progressive Disease (irPD) and Immune-related Stable Disease (irSD).

With respect to non-target lesions, responses to the treatment regimen described herein may include: Complete Response (CR), Progressive Disease (PD), Immune-related Complete Response (irCR) and Immune-related Progressive Disease (irPD).

In embodiments, the patient treated exhibits a complete response (CR), a partial response (PR), stable disease (SD), immune-related complete disease (irCR), immune-related partial response (irPR), or immune-related stable disease (irSD). In embodiments, the patient treated experiences tumor shrinkage and/or decrease in growth rate, i.e., suppression of tumor growth. In embodiments, unwanted cell proliferation is reduced or inhibited. In embodiments, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent.

Tumor response evaluation is performed using the following RECIST definitions.

The term “measurable disease” as used herein refers to presence of at least one measurable lesion. Measurable lesions must be accurately measured in at least 1 dimension (longest diameter in the plane of the measurement to be recorded) with a minimum size of:
10 mm by CT scan (CT scan slice thickness no greater than 5 mm).
10 mm caliper measurement by clinical exam (lesions which cannot be accurately measured with calipers should be recorded as non-measurable).
20 mm by chest X-ray.

The term “malignant lymph nodes” is defined to be pathologically enlarged and measurable, if a lymph node was ≥15 mm in short axis when assessed by CT scan (CT scan slice thickness recommended to be no greater than 5 mm). At baseline and in follow-up, only the short axis was measured and followed.

Non-Measurable Lesions

all other lesions, including small lesions (longest diameter <10 mm or pathological lymph nodes with ≥10 to <15 mm short axis), as well as non-measurable lesions.

The term “target lesions” refers to when more than one lesion is present at baseline all lesions up to a maximum of five lesions total representative of all involved organs are identified as target lesions. All other lesions including pathological lymph nodes are identified as “non-target lesions” and are also recorded at baseline.

The duration of overall response was measured from the time measurement criteria was first met for CR/PR (whichever was first recorded) until the first date that recurrent or PD was objectively documented (taking as reference for PD the smallest measurements recorded on study).

The duration of overall CR is measured from the time measurement criteria were first met for CR until the first date that recurrent disease was objectively documented.

Stable disease was measured from the start of the treatment until the criteria for progression were met, taking as reference the smallest sum on study (if the baseline sum was the smallest, this was the reference for calculation of PD).

For the evaluation of non-target lesions, the following definitions of the criteria were used to determine tumor response:

The term “complete response” or (CR) as used herein refers to disappearance of all non-target lesions and normalization of tumor marker level. All lymph nodes must be non-pathological in size (<10 mm short axis).

The term “non-CR/non-PD” as used herein refers to persistence of one or more non-target lesion(s) and/or maintenance of tumor marker level above the normal limits. The term “progressive disease” as used herein refers to unequivocal progression of existing non-target lesions.

The term “unequivocal progression” as used herein refers to an overall level of substantial worsening in non-target disease such that even in presence of SD or PR in target disease, the overall burden had increased sufficiently to merit discontinuation of therapy.

Progression Free Survival (PFS) is defined as the time from randomization to the date of documented disease progression or death, whichever occurs first, per RECIST 1.1 as assessed by the Investigator.

Overall Survival: is defined as the time from randomization to the date of death).

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

At various places in the present specification, substituents of compounds of the disclosure are disclosed in groups or in ranges. It is specifically intended that the disclosure include each and every individual sub combination of the members of such groups and ranges.

### SPECIFIC EMBODIMENTS OF THE PRESENT DISCLOSURE

Embodiment 1. A method of treating a sarcoma (e.g., soft tissue sarcoma and osteosarcoma), in a subject in need thereof, the method comprising administering to the subject an effective amount of Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

Embodiment 2: A method of enhancing an immune response in a subject suffering from a sarcoma (e.g. soft tissue sarcoma and osteosarcoma), the method comprising administering to said subject an effective amount of Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

Embodiment 3. A method of enhancing an innate immune response in a subject afflicted with sarcoma (e.g. soft tissue sarcoma and osteosarcoma), the method comprising administering to said subject, an effective amount of Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle and wherein the enhanced innate immune response is associated with increased tumoricidal natural killer cells and macrophages, as well as the activity of NK cells and CD8+ T cells.

Embodiment 4: A method of enhancing an innate immune response in a subject with sarcoma (e.g. soft tissue sarcoma and osteosarcoma), the method comprising administering to said subject an effective amount of Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle and wherein the enhanced innate immune response is associated with suppression of T-regulatory cells.

Embodiment 4: A method for treating, delaying progression or preventing or delaying tumor recurrence, tumor growth or spread of tumor in a subject afflicted with a sarcoma (e.g. soft tissue sarcoma and osteosarcoma), the method comprising administering to the subject an effective amount of Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

Embodiment 5: A method of enhancing immune function in a subject afflicted with a sarcoma (e.g. soft tissue sarcoma and osteosarcoma), the method comprising administering to the subject Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

Embodiment 6: A method for initiating, sustaining or enhancing an anti-tumor immune response in a subject afflicted with a sarcoma (e.g. soft tissue sarcoma and osteosarcoma), the method comprising administering to the subject an effective amount of Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

Embodiment 7: A method for reducing the treatment related adverse events (TRAEs) in a subject afflicted with a sarcoma (e.g. soft tissue sarcoma and osteosarcoma), comprising administering to a subject an effective amount of Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

Embodiment 8: The method of treatment according to any of Embodiments 1-7, wherein Talabostat or a pharmaceutically acceptable salt thereof and Pembrolizumab are administered to the subject in one or more (e.g. 1, 2, 3, 4, 5, 6 or more) treatment cycles, where each treatment cycle is of about 21 days duration.

Embodiment 9: The method of treatment according to any of Embodiments 1-8, wherein after cessation of treatment the subject maintains a sustained response to progression of sarcoma.

Embodiment 10: The method of treatment according to Embodiment 8 or 9, wherein for each treatment cycle Talabostat or a pharmaceutically acceptable salt thereof is administered on each of days 1 to 14 and Pembrolizumab is administered on day 1.

Embodiment 11: The method of treatment according to any of Embodiments 1-10, wherein Talabostat or a pharmaceutically acceptable salt thereof is administered orally (e.g. as a tablet formulation).

Embodiment 12: The method of treatment according to any of Embodiments 1-10, wherein Pembrolizumab is administered by injection (e.g. intravenously).

Embodiment 13: The method of treatment according to any of Embodiments 1-12, wherein Pembrolizumab is administered at a dose of from about 1 mg/kg to about 10 mg/kg per day.

Embodiment 14: The method of treatment according to any of Embodiments 1-13, wherein Talabostat or a pharmaceutically acceptable salt thereof is administered at a dose of about 0.3 mg in the morning and about 0.3 mg in the evening.

Embodiment 15: The method of treatment according to any of Embodiments 1-14, wherein Pembrolizumab is administered at a total dose of from about 100 mg to about 500 mg per day (e.g., about 200 mg per day).

Embodiment 16: The method of treatment according to any of Embodiments 1-14, wherein the total daily dose of Talabostat or a pharmaceutically acceptable salt thereof in cycle 1 is lower than the total daily dose of Talabostat or a pharmaceutically acceptable salt thereof in one or more subsequent cycles.

Embodiment 17: The method according to any of Embodiments 1 to 16, wherein the subject is administered Talabostat or a pharmaceutically acceptable salt thereof at a dose of about 0.2 mg twice daily for one or more consecutive days beginning on day 1 of the first treatment cycle.

Embodiment 18: The method according to any of Embodiments 1 to 17, wherein the subject is administered Talabostat or a pharmaceutically acceptable salt thereof at a dose of about 0.2 mg twice daily on days 1-7 of the first treatment cycle followed by about 0.3 mg twice daily on days 8-14 of the first treatment cycle.

Embodiment 19: The method according to any of Embodiments 1 to 7, wherein the subject is administered Talabostat or a pharmaceutically acceptable salt thereof at a dose of about 0.3 mg twice daily on days 1-3 of the first treatment cycle followed by rest period (day 4 to 7) and then about 0.3 mg twice daily (on days 8-11) of the first treatment cycle.

Embodiment 20: The method of treatment according to any of Embodiments 1 to 7, wherein the subject has an Eastern Cooperative Oncology Group (ECOG) performance status of 0-2.

Embodiment 21: The method according to any of Embodiments 1 to 7, wherein the subject experiences less treatment-related adverse events (TRAEs) relative to a subject with same caner and administered a single 0.6 mg once daily dose of Talabostat

Embodiment 22: The method according to Embodiment 21, wherein the subject experiences no TRAEs.

Embodiment 23: The method according to Embodiment 21 or 22, wherein the TRAE are one or more of hypotension, dizziness, headache, syncope, dyspnea, chills, pyrexia, malaise, weakness, edema/peripheral swelling, hypovolemia, hypothermia, fatigue, nausea, vomiting, diaphoresis, flushing, migraine, diarrhea, constipation, alopecia, pharyngitis, chest pain, anorexia, weight increase, weight decrease, vertigo, syncope, conjunctivitis, blurred vision, pallor, pruritus, rash, fungal vaginosis, hyperglycemia, hyperkalemia, hypokalemia, hoarseness, dyspnea, anoxia, deep venous thrombosis, upper respiratory infection, blood in stool, dizziness, rigors, sepsis, pain, hypereosinophilia, dehydration, electrolyte imbalance, arthralgia, rhabdomyolysis, myalgia, constipation, hypocalcemia, neutropenia, febrile neutropenia, anemia, leukopenia, pancytopenia, and lymphopenia, somnolence, insomnia, epistaxis, dyspepsia, dysgeusia, thrombocytopenia, cyanosis peripheral, hypovolemic shock, respiratory failure, cough, pneumonitis, cardiac tamponade, acidosis, renal failure and cardiac arrest.

Embodiment 24: The method according to any of preceding Embodiments, wherein the subject achieves a stable disease response or better, as measured by RECIST 1.1.

Embodiment 25: The method according to any of preceding Embodiments, wherein the subject achieves a partial response or better, as measured by RECIST 1.1.

Embodiments 26: The method of any of the preceding embodiments, wherein the subject achieves a complete response, as measured by RECIST 1.1.

Embodiment 27: The method according to any of preceding embodiments, wherein the subject experiences an increase in pro-inflammatory cytokines.

Embodiment 28: The method according to Embodiment 27, wherein the pro-inflammatory cytokines are one or more of IL-18 and IFN-γ.

Embodiment 29: The method according to any of Embodiments 1 to 7, wherein the subject was not previously treated with PD-1/PD-L1 or CTLA-4 antibodies.

Embodiment 30: The method according to any of Embodiments 1 to 6, wherein the subject has relapsed or progressed with PD-1/PD-L1 or CTLA-4 antibodies.

Embodiment 31. The method according to any of Embodiments 1 to 7, wherein subject has been previously treated with one or more regimens of systemic therapies for sarcoma.

Embodiment 32. The method according to any of Embodiments 1 to 7, wherein the subject has not been previously treated any systemic therapy for sarcoma.

Embodiment 33. The method according to any of Embodiments 1 to 7, wherein the subject has experienced no improvement from previous therapies.

Embodiment 34. The method according to any of Embodiments 1 to 7, wherein the subject has progressed or relapsed with systemic therapies for sarcoma.

Embodiment 35. The method according to any of Embodiments 1 to 7, wherein the subject experienced intolerable toxicity on or after systemic therapies for sarcoma.

Embodiment 36. The method according to any of Embodiments 1 to 7, wherein the subject is a human.

Embodiment 37. The method according to any of Embodiments 1 to 7, wherein the subject is not suffering from Ewing's sarcoma.

Embodiment 38. The method according to any of Embodiments 1 to 7, wherein the subject is not suffering from gastrointestinal stromal tumor.

Embodiment 39. The method according to any of Embodiments 1 to 7, wherein the subject is ≥12 years of age.

Embodiment 40: The method of treatment according to any of Embodiments 1-39, comprising administering an effective amount of Talabostat mesylate.

Embodiment 41: The method of treatment according to any of Embodiments 1-40, wherein Talabostat mesylate is administered orally as one or more tablets to provide a total daily dose of Talabostat of about 0.6 mg in divided doses and Pembrolizumab is administered as a single intravenous injection to provide a dose of from about 100 mg to about 500 mg per day.

Embodiment 42. The method according to Embodiment 41, wherein Talabostat or a pharmaceutically acceptable salt thereof is administered orally in the morning and evening.

Embodiment 43. The method according to Embodiment 41, wherein the subject experiences no treatment-related adverse events (TRAEs)

Embodiment 44. The method according to Embodiment 41, wherein the subject has a measurable disease as per RECIST 1.1 or iRECIST.

Embodiment 45. The method according to Embodiment 41, wherein the subject has a detectable bone metastases by whole body bone scintigraphy.

Embodiment 46. The method according to Embodiment 41, wherein the subject after the administration meets the CTC response as per Veridex assay.

Embodiment 47. The method according to Embodiment 41, wherein the subject achieves a stable disease response or betteras per RECIST 1.1.

Embodiment 48. The method according to Embodiment 41, wherein the subject achieves a complete or a partial response or better as per RECIST 1.1.

Embodiment 49. The method according to any of the preceding embodiments, wherein the sarcoma is advanced soft tissue sarcoma.

Embodiment 50. The method according to any of the preceding embodiments, wherein the soft tissue sarcoma is liposarcoma.

Embodiment 51. The method according to any of the preceding embodiments, wherein the liposarcoma is myxoid sarcoma.

Embodiment 51. A method for treating or inhibiting the growth of soft tissue sarcoma in a subject in need thereof, comprising administering to said subject, Talabostat or a pharmaceutically acceptable salt thereof at a dose of about 0.3 mg twice daily on one or more days of a treatment cycle and Pembrolizumab on day 1 of the treatment cycle.

Embodiment 52. A method for treating or inhibiting the growth of soft tissue sarcoma in a subject in need thereof, comprising administering to said subject, Talabostat or a pharmaceutically acceptable salt thereof orally at a dose of about 0.3 mg twice daily on day 1-3 of the first treatment cycle followed by rest period of 4 days then about 0.3 mg twice daily on day 8-11 of the first treatment cycle and Pembrolizumab intravenously at a total dose of about 200 mg.

Embodiment 53. A method for treating or inhibiting the growth of soft tissue sarcoma in a subject in need thereof, comprising administering to said subject, Talabostat or a pharmaceutically acceptable salt thereof orally at a dose of about 0.2 mg twice daily on day 1-7 of the first treatment cycle followed by about 0.3 mg twice daily on day 8-14 of the first treatment cycle and Pembrolizumab intravenously on day 1 of the treatment cycle.

Embodiment 54. A method for treating or inhibiting the growth of liposarcoma in a subject in need thereof, comprising administering to said subject, Talabostat or a pharmaceutically acceptable salt thereof at a dose of about 0.3 mg twice daily on one or more days of a treatment cycle and Pembrolizumab on day 1 of the treatment cycle.

Embodiment 55. A method for treating or inhibiting the growth of liposarcoma in a subject in need thereof, comprising administering to said subject, Talabostat or a pharmaceutically acceptable salt thereof orally at a dose of about 0.3 mg twice daily on day 1-3 of the first treatment cycle followed by rest period of 4 days then about 0.3 mg twice daily on day 8-11 of the first treatment cycle and Pembrolizumab intravenously at a total dose of about 200 mg.

Embodiment 56. A method for treating or inhibiting the growth of liposarcoma in a subject in need thereof, comprising administering to said subject, Talabostat or a pharmaceutically acceptable salt thereof orally at a dose of about 0.2 mg twice daily on day 1-7 of the first treatment cycle followed by about 0.3 mg twice daily on day 8-14 of the first treatment cycle and Pembrolizumab intravenously on day 1 of the treatment cycle.

Embodiment 57. A method for treating or inhibiting the growth of myxoid sarcoma in a subject in need thereof, comprising administering to said subject, Talabostat or a pharmaceutically acceptable salt thereof at a dose of about 0.3 mg twice daily on one or more days of a treatment cycle and Pembrolizumab on day 1 of the treatment cycle.

Embodiment 58. A method for treating or inhibiting the growth of myxoid sarcoma in a subject in need thereof, comprising administering to said subject, Talabostat or a pharmaceutically acceptable salt thereof orally at a dose of about 0.3 mg twice daily on day 1-3 of the first treatment cycle followed by rest period of 4 days then about 0.3 mg twice daily on day 8-11 of the first treatment cycle and Pembrolizumab intravenously at a total dose of about 200 mg.

Embodiment 59. A method for treating or inhibiting the growth of myxoid sarcoma in a subject in need thereof, comprising administering to said subject, Talabostat or a pharmaceutically acceptable salt thereof orally at a dose of about 0.2 mg twice daily on day 1-7 of the first treatment cycle followed by about 0.3 mg twice daily on day 8-14 of the first treatment cycle and Pembrolizumab intravenously on day 1 of the treatment cycle.

### Examples

Example 1: Study of Talabostat mesylate, a small molecule inhibitor of Dipeptidyl peptidase (DPP) administered in combination with Anti-Programmed cell death 1 (PD-1) Monoclonal antibody Pembrolizumab (PEMBRO, Keytruda®) in patients with advanced soft tissue sarcomas (STS).

Objectives:

Primary:

To evaluate the objective response rate of Talabostat mesylate, single-agent, Pembrolizumab single-agent, and the combination of Talabostat mesylate with Pembrolizumab, in patients with advanced STS, defined as the percentage of patients with confirmed complete or partial response, per Response Evaluation Criteria in Solid Tumors (RECIST) version 1.1.

Secondary:

To evaluate the following outcome measures for Talabostat mesylate single-agent, pembrolizumab single-agent, and the combination of Talabostat mesylate with pembrolizumab treatment groups:
1. Clinical benefit rate (CBR), defined as the percentage of patients with confirmed CR or PR or with stable disease lasting for ≥16 weeks
2. Progression-free survival (PFS), defined as the time from randomization to the date of documented disease progression or death, whichever occurs first
3. Overall survival (OS), defined as the time from randomization to the date of death
4. Safety, based on the incidence of adverse events, laboratory abnormalities, and dose reductions

Exploratory
1. Cell-free DNA (cfDNA) analyses over time
2. Evaluate quantitative changes in the systemic immune system
3. Assess the predictive value of markers associated with PD-(L)1 therapy and DPP4/DPP8/DPP9 tumor expression and amplification, macrophage density and other potential predictive markers associated with outcomes of treatment with Talabostat mesylate or Talabostat mesylate plus pembrolizumab in baseline and optionally in on-treatment tumor samples.
4. Other sarcoma-related markers
5. Contribute to the population pharmacokinetics assessment of Talabostat mesylate using sparse pharmacokinetic sampling
6. Assess the clinical outcomes in patients following crossover from assigned single agent arm to combination therapy.

Population

Patients with one of the three most common types of STS (leiomyosarcoma, liposarcoma or undifferentiated pleomorphic sarcoma) plus a fourth cohort of patients with other types of STS are entered in the study.

Specific Inclusion/Exclusion Criteria:

Inclusion criteria:
1. Age ≥12 years of age at the time of informed consent if local regulations and/or institutional policies allow for participants <18 years of age with consent of parents or guardians plus assent of participant. If not allowed, then ≥18 years applied. Patients <18 years of age must weigh ≥40 kgs.
2. ECOG PS≤2
3. Histologically confirmed surgically unresectable or metastatic soft tissue sarcoma excluding Ewings Sarcoma and GIST.
4. Patient has a measurable disease by CT/MRI per RECIST 1.1
5. Patient has one or two regimens of systemic therapy for the sarcoma with disease progression or intolerable toxicity on or after last regimen and has no satisfactory treatment options. (Neo)adjuvant and maintenance treatments did not count toward this criterion.
6. Patient agreed to pre-treatment fresh core, excision or punch tumor biopsy for biomarker analysis, if accessible and low risk. On-treatment biopsy is optional.
7. Screening blood counts as follows:
   a. Absolute neutrophil count ≥1500/μL
   b. Platelets ≥100,000/μL and no platelet transfusions during the prior 14 days.
   c. Hemoglobin ≥8 g/dL and no red blood cell transfusions during the prior 14 days.
8. Screening chemistry as follows:
   a. Alanine aminotransferase (ALT) and aspartate transaminase (AST) ≤3×upper limit of the normal reference range (ULN)
   b. Total bilirubin ≤1.5×ULN
   c. Serum creatinine ≤1.5×ULN or calculated Cr clearance >50 mL/min.
   d. Albumin >3.0 g/dL
9. Women of childbearing potential (WOCBP) and men must agree to use effective contraceptive methods until at least 6 months following the last dose of study medication.
10. WOCBP must have a negative pregnancy test (serum or urine)
11. Participant has signed informed consent prior to any protocol-related procedures that are not part of normal patient care.
12. Minors who are judged to be of an age of reason as determined by local requirements must also give their assent. The assent should be documented based on local requirement. Continued assent should be documented when important new information becomes available that is relevant to the participant's assent.
13. Participant is able to adhere to the study visit schedule and other protocol requirements, including follow-up.

Exclusion Criteria:
1. Prior therapy with any anti-PD-1 or anti-PD-L1 antibody
2. Presence of other malignancy or history of other malignancy within 3 years, other than locally curable cancers that has been apparently cured Patient has received systemic therapy or external beam radiation within 14 days of randomization. If lesions are progressing despite prior therapy, treatment on this study might begin 2 weeks after prior systemic therapy or external beam radiation.
3. Patient has not fully recovered from reversible toxicity of prior systemic therapy
4. History of symptomatic orthostatic hypotension within 3 months prior to enrollment. Orthostatic hypotension is defined as a drop in systolic blood pressure (BP) of ≥20 mmHg or diastolic BP of ≥10 mmHg with assumption of an upright posture.
5. Known gastrointestinal disease which may affect absorption of study medication
6. Inability at baseline to swallow capsules/tablets
7. Patient has clinically significant cardiovascular disease (e.g., uncontrolled or any New York Heart Association Class 3 or 4 congestive heart failure, uncontrolled angina, history of myocardial infarction, unstable angina or stroke within 6 months prior to study entry, uncontrolled hypertension, or clinically significant arrhythmias not controlled by medication).
8. Other serious concurrent medical, psychiatric or social condition which placed the patient at an unacceptably high risk for toxicity
9. Receipt of investigational medication for any condition within the 4 weeks prior to planned start of study medication
10. Patient has QT interval corrected for heart rate using Bazett's formula (QTcB) >480 msec at Screening.
11. Patient has uncontrolled, clinically significant pulmonary disease (e.g., chronic obstructive pulmonary disease, pulmonary hypertension) that in the opinion of the investigator would put the patient at significant risk for pulmonary complications during the study.
12. Patient has brain or leptomeningeal metastases that are symptomatic and progressive on imaging. Patients with a history of CNS metastases must have received appropriate treatment. Central nervous system imaging is not required prior to study entry unless there is a history of CNS involvement or clinical suspicion of CNS involvement. Imaging of patients with a prior history of CNS metastases should be compared to prior imaging to discern PD.
13. Patient has an active autoimmune disease or Grade ≥3 (non-infectious) pneumonitis that has required systemic treatment in the past 2 years (i.e., with use of disease modifying agents, corticosteroids, or immunosuppressive drugs). Replacement therapy (e.g., thyroxine, insulin, or physiologic corticosteroid replacement therapy for adrenal or pituitary insufficiency) or treatment with drugs (e.g., neomercazol, carbimazole) that function to decrease the generation of thyroid hormone by a hyperfunctioning thyroid gland (e.g., in Graves' disease) is not considered a form of systemic treatment of an autoimmune disease.
14. Patient require concomitant treatment with gliptins, or systemic corticosteroids >10 mg daily prednisone/equivalent) or other immunosuppressive medications with 14 days of study drug initiation
15. Patient has uncontrolled intercurrent illness including, but not limited to, uncontrolled infection, suspected or active SARS-CoV-2 (Covid-19) infection, disseminated intravascular coagulation, or psychiatric illness/social situations that would limit compliance with study requirements
16. Patient has known positive status for human immunodeficiency virus, active or chronic Hepatitis B, or Hepatitis C. Screening is not required

Design

The study apply an enrichment strategy by having an initial non-randomized lead-in phase to determine which histologic subtype are further evaluated in the randomized phase of the study.

Lead-In Phase

Patients are enrolled and classified into one of the four histologic diagnosis subtypes: leiomyosarcoma, liposarcoma, undifferentiated pleomorphic sarcoma, all other STS. Up to 11 patients are enrolled in each histologic subtype and treated with Talabostat mesylate and Pembrolizumab in combination. If ≥2 CBR responses are observed (i.e., if at least 2 patients has confirmed PR or CR or have stable disease lasting at least 16 weeks), then the histologic subtype is included in the randomized phase of the study. Otherwise, the subtype is excluded for lack of efficacy.

This decision rule is consistent with the futility stopping rule for the first stage of a Simon two-stage optimal design under a null hypothesis that the CBR response probability is ≤10%, an alternative hypothesis that the CBR response probability is ≥30%, a one-sided type 1 error rate of 0.05, and power=85%. As soon as a subtype met the threshold of ≥2 CBR responses, the subtype is proceeded to enrollment in the randomized phase.

Randomized Phase

Each histologic subtype in the lead-in phase that met the criterion for continued evaluation is included in the randomized component of the study. Subtype enrollment in the randomized phase might occur in a staggered manner depending on the enrollment rate in the lead-in phase.

Patients are randomized 1:1:1, stratified by histologic subtype, to receive one of the following three treatment regimens:
Talabostat mesylate alone;
Pembrolizumab alone; or
Talabostat mesylate and pembrolizumab in combination.

The total sample size depend on the number of histologic subtypes that proceeded to the randomized phase of the study.
Two subtypes in the randomized phase: A maximum of 60 patients are enrolled for each subtype, and the total sample size is approximately 120 patients.
Three subtypes in the randomized phase: A maximum of 45 patients are enrolled for each subtype, and the total sample size is approximately 135 patients.
All four subtypes in the randomized phase: A maximum of 36 patients are enrolled for each subtype, and the total sample size is approximately 144 patients.

Efficacy outcomes by treatment arm are estimated across and within histologic subtypes.

Patients are continued on assigned therapy until documented disease progression or development of unacceptable toxicity. Patients assigned to single agent treatment discontinuing for protocol defined progression may continue treatment beyond progression, cross-over to combination therapy or discontinue all study treatment. Patients discontinuing study therapy for unacceptable toxicity are not permitted to cross-over and should have all end of treatment procedures performed.

Patients are followed for response and progression by physical examination and imaging; they are followed for toxicity by physical examination, reported adverse events, and changes in laboratory examinations.

Methodology

After consent, qualification and stratification, patients are randomized to receive one of the following three treatment regimens:
1. Talabostat mesylate alone: 0.2 mg po BID for the first week of treatment in Cycle 1, with escalation to 0.3 mg po BID if the lower dose is well tolerated. Dosing occurs on day 1-14 of every 21 day cycle. Treatment is to continue until documented disease progression or occurrence of unacceptable toxicity not resolved by dose reduction.
2. Pembrolizumab alone: 200 mg IV infusion over 30 minutes every 21 days until disease progression or occurrence of unacceptable. The recommended dose in adolescents is 2 mg/kg body weight (up to a maximum of 200 mg) administered as an IV infusion over ≥30 minutes. Premedication according to standard local practices is permitted.
3. Talabostat mesylate plus pembrolizumab: each drug is given at full dose, as described above.

Endpoints

Efficacy:

Primary endpoint: Objective response rate (ORR), defined as the percentage of patients with confirmed complete response (CR) or partial response (PR), per RECIST1.1 as assessed by the Investigator

Secondary Endpoints:

1. Clinical benefit rate (CBR), defined as the percentage of patients with confirmed CR or PR or with stable disease lasting ≥16 weeks, per RECIST 1.1 as assessed by the Investigator Progression-free survival (PFS), defined as the time from randomization to the date of documented disease progression or death, whichever occurs first, per RECIST 1.1 as assessed by the Investigator. Patients who are alive and progression-free at the time of analysis are censored on the date of the last tumor assessment. Patients who started subsequent anti-cancer therapy prior to documented progression are censored on the date of the last tumor assessment on or prior to the start date of the subsequent anti-cancer therapy. OS is defined as the time from randomization to the date of death. Patients who are alive at the time of analysis are censored on the date of last contact (“last known alive date”).

2. Pharmacokinetics: Samples are collected to contribute to the estimation of PK parameters such as maximum concentration (Cmax), time to Cmax (Tmax), area under the curve (AUC), clearance, and half-life (t½)

Safety: Rate of AEs, SAES, AEs leading to dose reductions, AEs leading to discontinuation of treatment, deaths and laboratory abnormalities

Exploratory
1. Changes in cell-free DNA (cfDNA) total levels and components over time
2. Immune markers over time: Pre and on-treatment changes in levels of select immune cells and cytokines in the systemic circulation
3. In tumor, pre-treatment and optionally on-treatment biomarker levels and changes in tumor PD-L1 expression, tumor mutation burden, microsatellite status; tumor DPP4/DPP8/DPP9 expression and amplification, macrophage density and other potential predictive markers associated with outcomes of treatment; cellular composition of the TME; Gene expression changes in the TME
4. Other sarcoma-related markers
5. Assess the clinical outcomes in patients following crossover from assigned single agent arm to combination therapy.

Statistical Methods

Sample Size Rationale

The total sample size depends on the number of histologic subtypes that proceeded to the randomized phase of the study.
Two subtypes proceed to the randomized phase: A maximum of 60 patients are enrolled for each subtype, and the total sample size is approximately 120 patients (40 per arm).
Three subtypes proceed to the randomized phase: A maximum of 45 patients are enrolled for each subtype, and the total sample size is approximately 135 patients (45 per arm).
All four subtypes proceed to the randomized phase: A maximum of 36 patients are enrolled for each subtype, and the total sample size is approximately 144 patients (48 per arm).

Efficacy outcomes by treatment arm is estimated across histologic subtypes, with sensitivity analyses performed by individual subtype. The table 4 below provided 80% confidence intervals (CIs) for the difference in ORR between the combination treatment of Talabostat mesylate plus pembrolizumab and single-agent pembrolizumab under varying benchmark assumptions if the sample size is 40-48 patients per arm (120-144 patients total). The ORR in the combination arm provided the effect size necessary to rule out a difference of 0; in all cases, the target ORR in the combination arm is that which yielded an absolute ORR difference of 12.5 percentage points.

TABLE 4

| Sample size | Pembrolizumab | Combination | | Difference |
|---|---|---|---|---|
| per arm | ORR | ORR | | (80% CI^{a}) |
| 40 | 17.5% (7/40) | 30.0% | (12/40) | 12.5 (0.23, 24.6) |
| | 15.0% (6/40) | 27.5% | (11/40) | 12.5 (0.72, 24.2) |
| | 10.0% (4/40) | 22.5% | (8/40) | 12.5 (1.93, 23.3) |
| 48 | 18.8% (9/48) | 31.3 | (15/48) | 12.5 (0.01, 24.8) |
| | 14.6% (7/48) | 27.1 | (13/48) | 12.5 (0.80, 24.2) |
| | 10.4% (5/48) | 22.9 | (11/48) | 12.5 (1.82, 23.4) |

^{a}Miettinen and Nurminen (1985) - PASS v15.0.1

### Statistical Analyses

Tumor response assessments are based on RECIST 1.1 using the Investigator's assessment. All efficacy analyses are performed for all randomized patients across all histologic subtypes. Sensitivity analyses by subtype is also conducted.

To evaluate proof-of-concept for an efficacy signal, a two-sided 80% exact CI as well as a 95% exact CI is calculated for ORR using the Miettinen-Nurminen method. Best response is summarized by response category. In addition, duration of response (DOR) is estimated. DOR is measured from the time RECIST criteria are first met for CR or PR and confirmed at least 4 weeks later until the date of documented progression or death, whichever occurred first. The DOR censoring rules are the same as the PFS censoring rules.

CBR also contributed to evaluation of efficacy proof-of-concept. A two-sided 80% exact CI as well as a 95% exact CI is calculated for CBR using the Clopper-Pearson method.

DOR, PFS, and OS are estimated using the Kaplan-Meier (KM) product limit method. The KM estimate of the medians for each of these endpoints are reported by treatment group, and the corresponding 95% CI are computed using Brookmeyer and Crowley's method (log-log transformation). KM estimates of PFS and OS rates at milestone time points are reported for each treatment group along with two-sided 95% CIs using Greenwood's formula, provided the minimum follow-up in patients exceeded the time point to support stable estimation of the rate.

Safety evaluations are based on the incidence, intensity, and type of treatment-emergent adverse event (TEAE) or serious adverse event (SAE), as well as changes in safety assessments. TEAEs leading to treatment discontinuation are also summarized.

Safety outcomes including TEAEs and laboratory evaluations are summarized using descriptive statistics for all patients who received at least 1 dose of study drug.

AEs are assessed for severity according to the CTCAE Version 5.0 (CTCAE 2017); verbatim AE terms are coded using the Medical Dictionary for Regulatory Activities (MedDRA) for purposes of summarization.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

What is claimed is:

1. A method of treating a soft tissue sarcoma in a subject, comprising administering to the subject an effective amount of Talabostat or a pharmaceutically acceptable salt thereof in combination with a therapeutically effective amount of Pembrolizumab, wherein Talabostat or a pharmaceutically acceptable salt thereof is administered at a dose of about 0.3 mg twice daily on one or more days of a treatment cycle.

2. A method of treating liposarcoma in a subject, comprising administering to the subject an effective amount of Talabostat or a pharmaceutically acceptable salt thereof in combination with an effective amount of Pembrolizumab, wherein Talabostat or a pharmaceutically acceptable salt thereof is administered at a dose of about 0.3 mg twice daily on one or more days of a treatment cycle.

3. A method of treating myxoid cancer in a subject, comprising administering to the subject an effective amount of Talabostat or a pharmaceutically acceptable salt thereof in combination with an effective amount of Pembrolizumab, wherein Talabostat or a pharmaceutically acceptable salt thereof is administered at a dose of about 0.3 mg twice daily on one or more days of a treatment cycle.

4. The method of claim 1, wherein the subject is not previously treated with PD-1/PD-L1 or CTLA-4 antibodies.

5. The method of claim 1, wherein the subject has relapsed or progressed with PD-1/PD-L1 or CTLA-4 antibodies.

6. The method of claim 1, wherein the treatment cycle is a 21-day treatment cycle and Talabostat or a pharmaceutically acceptable salt thereof is administered on each of days 1 to 14 and Pembrolizumab is administered on day 1.

7. The method of claim 1, wherein the subject is administered Talabostat or a pharmaceutically acceptable salt thereof at a dose of about 0.2 mg twice daily for one or more consecutive days beginning on day 1 of the first treatment cycle.

8. The method of claim 7, wherein the subject is administered Talabostat or a pharmaceutically acceptable salt thereof at a dose of about 0.2 mg twice daily on days 1-7 of the first treatment cycle followed by about 0.3 mg twice daily on days 8-14 of the first treatment cycle.

9. The method of claim 1, comprising one or more additional treatment cycles.

10. The method of claim 1, wherein Talabostat or a pharmaceutically acceptable salt thereof is administered orally in the morning and evening.

11. The method of claim 1, wherein Talabostat or a pharmaceutically acceptable salt thereof is present as Talabostat mesylate.

12. The method of claim 1, wherein Pembrolizumab is administered intravenously at a total dose of about 200 mg.

13. The method of claim 1, wherein the subject experiences no TRAEs selected from hypotension, dizziness, headache, syncope, dyspnea, chills, pyrexia, malaise, weakness, edema/peripheral swelling, hypovolemia, hypothermia, fatigue, nausea, vomiting, diaphoresis, flushing, migraine, diarrhea, constipation, alopecia, pharyngitis, chest pain, anorexia, weight increase, weight decrease, vertigo, syncope, conjunctivitis, blurred vision, pallor, pruritus, rash, fungal vaginosis, hyperglycemia, hyperkalemia, hypokalemia, hoarseness, dyspnea, anoxia, deep venous thrombosis, upper respiratory infection, blood in stool, dizziness, rigors, sepsis, pain, hypereosinophilia, dehydration, electrolyte imbalance, arthralgia, myalgia, constipation, hypocalcemia, neutropenia, febrile neutropenia, anemia, leukopenia, pancytopenia, and lymphopenia, somnolence, insomnia, epistaxis, dyspepsia, dysgeusia, thrombocytopenia, cyanosis peripheral, hypovolemic shock, respiratory failure, cough, pneumonitis, cardiac tamponade, acidosis, renal failure and cardiac arrest.

14. The method of claim 1, wherein the subject achieves a stable disease response or better, as measured by RECIST 1.1.

15. The method of claim 1, wherein the subject achieves a partial response or better, as measured by RECIST 1.1.

16. The method of claim 1, wherein the subject achieves a complete response, as measured by RECIST 1.1.

17. The method of claim 1, wherein the soft tissue sarcoma is liposarcoma.

18. The method of claim 1, wherein the soft tissue sarcoma is myxoid cancer.
